(19) United States
(12) Patent Application Publication
(10) Pub. No.: US 2026/0226090 A1
(43) Pub. Date: Aug. 6, 2026

(54) SODIUM -GLUCOSE CONTRANSPORTER 2 INHIBITOR DERIVATIVE

(71) Applicant: Anxo Pharmaceutical Co., Ltd., Taipei City (TW)

(72) Inventors: Te-I LIU, Taipei City (TW); Chia-Yu SU, Taipei City (TW); Sheng-Hung LIU, Taipei City (TW); Yi-Hsiang LIU, Taipei City (TW); Hua-Jing JHAN, Taipei City (TW); Chi-Heng JIAN, Taipei City (TW)

(21) Appl. No.: 19/465,514

(22) Filed: Jan. 30, 2026

Related U.S. Application Data
(60) Provisional application No. 63/754,583, filed on Feb. 6, 2025.

(51) Int. Cl. C07H 7/04; C07H 7/06

(52) U.S. Cl. CPC C07H 7/04; C07H 7/06

(57) ABSTRACT

In some embodiments of the present disclosure, a sodium-glucose cotransporter 2 (SGLT2) inhibitor derivative is provided, comprising a backbone structure and a hydrophobic functional group, in which the backbone structure comprises a glucoside or a glucoside analog, in which the glucoside or the glucoside analog is bonded to the hydrophobic functional group via a covalent bond.

### CROSS-REFERENCE TO RELATED APPLICATION

This application claims priority to U.S. Provisional Application Ser. No. 63/754,583, filed Feb. 6, 2025, which was herein incorporated by reference in its entirety.

### BACKGROUND

### Field of Invention

The present invention is about a sodium-glucose cotransporter 2 (SGLT2) inhibitor derivative. In particular, the present invention was about a SGLT2 inhibitor derivative with hydrophobic functional group.

### Description of Related Art

Sodium-glucose cotransporter 2 (SGLT2) mostly expresses in proximal tubular cells and is used to reabsorb glucose in the kidneys, thereby promoting glucose to pass through kidney cells and then be absorbed into the blood. SGLT2 inhibitor is known to be used for diabetes, chronic kidney disease and heart failure treatment, and there are several dosage forms, for example, Forxiga®, Canaglu®, Jardiance® and Steglatro®.

However, current SGLT2 inhibitors are only used to prepare short-acting oral dosage forms, and there are no SGLT2 inhibitors suitable for long-term treatment.

Therefore, how to provide a SGLT2 inhibitor derivative to improve the above mentioned problem remains to be solved.

### SUMMARY

In one aspect of the present disclosure, a sodium-glucose cotransporter 2 (SGLT2) inhibitor derivative is provided, comprising a backbone structure and a hydrophobic functional group, in which the backbone structure comprises a glucoside or a glucoside analog, in which the glucoside or the glucoside analog is bonded to the hydrophobic functional group via a covalent bond.

In some embodiments, the backbone structure comprises a structure as below:

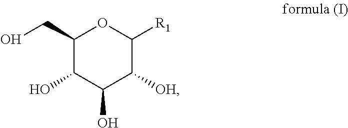

in which R₁ represents an unsubstituted aryl group, a substituted aryl group, an arylalkyl group, an arylalkylenearyl group, or a heteroaryl group, in which a substituent of the substituted aryl group comprises C₁-C₆ alkyl, halo, alkoxy, alkenyl or heteroaryl.

In some embodiments, the glucoside comprises Velagliflozin, Dapagliflozin, Canagliflozin, Empagliflozin, Tofogliflozin, Ipragliflozin, Atigliflozin, Bexagliflozin or Janagliflozin.

In some embodiments, the glucoside analog comprises a first glucoside analog, a second glucoside analog or a third glucoside analog, in which the first glucoside analog represents oxygen-containing bridge skeleton formed in glucose six-membered ring of the glucoside, the second glucoside analog represents that oxygen atom in the glucose six-membered ring of the glucoside is replaced by sulfur, and the third glucoside analog represents carbon 6 in the glucoside is replaced by a methylthio group.

In some embodiments, the first glucoside analog comprises a structure as below:

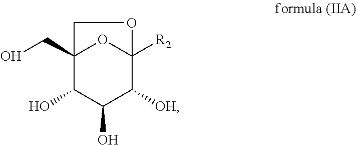

in which R₂ represents an unsubstituted aryl group, a substituted aryl group, an arylalkyl group, an arylalkylenearyl group, or a heteroaryl group, in which a substituent of the substituted aryl group comprises C₁-C₆ alkyl, halo, alkoxy, alkenyl or heteroaryl.

In some embodiments, the first glucoside analog comprises Ertugliflozin or Henagliflozin.

In some embodiments, the second glucoside analog comprises a structure as below:

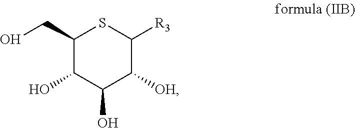

wherein R₃ represents an unsubstituted aryl group, a substituted aryl group, an arylalkyl group, an arylalkylenearyl group, or a heteroaryl group, wherein a substituent of the substituted aryl group comprises C₁-C₆ alkyl, halo, alkoxy, alkenyl or heteroaryl.

In some embodiments, the second glucoside analog comprises Luseogliflozin.

In some embodiments, the third glucoside analog comprises a structure as below:

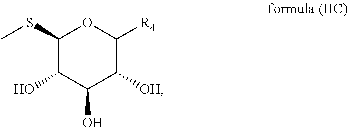

in which R₄ represents an unsubstituted aryl group, a substituted aryl group, an arylalkyl group, an arylalkylenearyl group, or a heteroaryl group, in which a substituent of the substituted aryl group comprises C₁-C₆ alkyl, halo, alkoxy, alkenyl or heteroaryl.

In some embodiments, the third glucoside analog comprises Sotagliflozin.

In some embodiments, the hydrophobic functional group comprises an aliphatic group, an aromatic ring group or a combination thereof, in which the aliphatic group has at least 6 carbon atoms.

In some embodiments, the hydrophobic functional group is bonded at oxygen atom of C2, C3, C4, or C6 of the glucoside or the glucoside analog.

In some embodiments, when the backbone structure is the glucoside, the SGLT2 inhibitor derivative has a structure of formula (III):

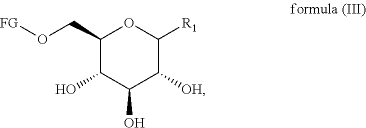

in which when the backbone structure is the glucoside analog, the SGLT2 inhibitor derivative has a structure of formula (IVA), formula (IVB) or formula (IVC):

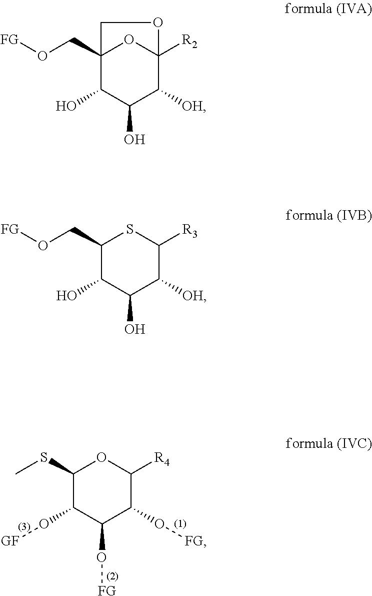

in which R₁, R₂, R₃ and R₄ represent an unsubstituted aryl group, a substituted aryl group, an arylalkyl group, an arylalkylenearyl group, or a heteroaryl group, respectively, a substituent of the substituted aryl group comprises C₁-C₆ alkyl, halo, alkoxy, alkenyl or heteroaryl, and FG represents the hydrophobic functional group and is bonded to O at position (1), position (2), position (3) or a combination thereof.

In some embodiments, the aliphatic group comprises hexyl, octyl, decyl, dodecyl, tetradecyl, hexadecyl, octadecyl, icosyl, docosyl, tetracosyl, caproyl, capryloyl, decanoyl, dodecanoyl, tetradecanoyl, hexadecanoyl, octadecanoyl, icosanoyl, docosanoyl, tetracosanoyl or a combination thereof.

In some embodiments, the aromatic ring group comprises cinnamoyl, phenylacetyl, indanyl, salicyl, anthranilyl, benzoyl or a combination thereof.

In some embodiments, the covalent bond comprises an ester bond, an acetal bond or a ketal bond.

In some embodiments, the ester bond is a carbonate ester bond, a fatty acid ester bond, a phosphodiester bond, an aromatic ester bond or a carbamate bond.

In some embodiments, the SGLT2 inhibitor derivative has a hydrophobic constant of more than 3.

In some embodiments, the hydrophobic constant is calculated by X Log P3 or measured by a distribution coefficient test of n-octanol-water system.

In some embodiments, a solubility of the SGLT2 inhibitor derivative is more than 0 and less than 0.1 mg/mL in pH 6 phosphate buffer.

### BRIEF DESCRIPTION OF THE DRAWINGS

In order to make the above-mentioned and other objects, features, advantages and embodiments of the present disclosure more clearly understood, descriptions of accompanying drawings are as follows:

FIG. 1 illustrates synthesis diagram of dapagliflozin palmitate (hexadecyl) ester derivative of some embodiments of the present disclosure.

FIG. 2 illustrates ¹H-NMR (proton nuclear magnetic resonance) spectrum of dapagliflozin palmitate (hexadecyl) ester derivative of some embodiments of the present disclosure.

FIG. 3 illustrates LC-MS (liquid chromatography-mass spectrometry) spectrum of dapagliflozin palmitate (hexadecyl) ester derivative of some embodiments of the present disclosure.

FIG. 4 illustrates synthesis diagram of dapagliflozin hexyl carbonate ester derivative of some embodiments of the present disclosure.

FIG. 5 illustrates ¹H-NMR spectrum of dapagliflozin hexyl carbonate ester derivative of some embodiments of the present disclosure.

FIG. 6 illustrates LC-MS spectrum of dapagliflozin hexyl carbonate ester derivative of some embodiments of the present disclosure.

FIG. 7 illustrates synthesis diagram of dapagliflozin aromatic ester derivative of some embodiments of the present disclosure.

FIG. 8 illustrates ¹H-NMR spectrum of dapagliflozin aromatic ester derivative of some embodiments of the present disclosure.

FIG. 9 illustrates LC-MS spectrum of dapagliflozin aromatic ester derivative of some embodiments of the present disclosure.

FIG. 10 illustrates synthesis diagram of empagliflozin palmitate (hexadecyl) ester derivative of some embodiments of the present disclosure.

FIG. 11 illustrates ¹H-NMR spectrum of empagliflozin palmitate (hexadecyl) ester derivative of some embodiments of the present disclosure.

FIG. 12 illustrates LC-MS spectrum of empagliflozin palmitate (hexadecyl) ester derivative of some embodiments of the present disclosure.

FIG. 13 illustrates synthesis diagram of empagliflozin hexyl carbonate ester derivative of some embodiments of the present disclosure.

FIG. 14 illustrates ¹H-NMR spectrum of empagliflozin hexyl carbonate ester derivative of some embodiments of the present disclosure.

FIG. 15 illustrates LC-MS spectrum of empagliflozin hexyl carbonate ester derivative of some embodiments of the present disclosure.

FIG. 16 illustrates synthesis diagram of empagliflozin aromatic ester derivative of some embodiments of the present disclosure.

FIG. 17 illustrates ¹H-NMR spectrum of empagliflozin aromatic ester derivative of some embodiments of the present disclosure.

FIG. 18 illustrates LC-MS spectrum of empagliflozin aromatic ester derivative of some embodiments of the present disclosure.

FIG. 19 illustrates synthesis diagram of bexagliflozin palmitate (hexadecyl) ester derivative of some embodiments of the present disclosure.

FIG. 20 illustrates ¹H-NMR spectrum of bexagliflozin palmitate (hexadecyl) ester derivative of some embodiments of the present disclosure.

FIG. 21 illustrates LC-MS spectrum of bexagliflozin palmitate (hexadecyl) ester derivative of some embodiments of the present disclosure.

FIG. 22 illustrates synthesis diagram of bexagliflozin hexyl carbonate ester derivative of some embodiments of the present disclosure.

FIG. 23 illustrates ¹H-NMR spectrum of bexagliflozin hexyl carbonate ester derivative of some embodiments of the present disclosure.

FIG. 24 illustrates LC-MS spectrum of bexagliflozin hexyl carbonate ester derivative of some embodiments of the present disclosure.

FIG. 25 illustrates synthesis diagram of bexagliflozin aromatic ester derivative of some embodiments of the present disclosure.

FIG. 26 illustrates ¹H-NMR spectrum of bexagliflozin aromatic ester derivative of some embodiments of the present disclosure.

FIG. 27 illustrates LC-MS spectrum of bexagliflozin aromatic ester derivative of some embodiments of the present disclosure.

FIG. 28 illustrates synthesis diagram of dapagliflozin hexyl ester derivative and docosyl ester derivative of some embodiments of the present disclosure.

FIG. 29 illustrates ¹H-NMR spectrum of dapagliflozin hexyl ester derivative of some embodiments of the present disclosure.

FIG. 30 illustrates LC-MS spectrum of dapagliflozin hexyl ester derivative of some embodiments of the present disclosure.

FIG. 31 illustrates ¹H-NMR spectrum of dapagliflozin docosyl ester derivative of some embodiments of the present disclosure.

FIG. 32 illustrates LC-MS spectrum of dapagliflozin docosyl ester derivative of some embodiments of the present disclosure.

### DETAILED DESCRIPTION

In order that the present disclosure was described in detail and completeness, implementation aspects and specific embodiments of the present disclosure with illustrative description are presented, but those are not the only form for implementation or use of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

Although a series of operations or steps are described below to illustrate the method disclosed herein, the order of the operations or steps was not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein may include a plurality of sub-steps or actions.

### Definition

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

As used herein, “glucoside analog” refers to a glucoside analog which is a compound structurally similar to a glucoside, in which variations are made to the saccharide moiety, the glycosidic linkage, or the aglycone part, while retaining the ability to interact with biological targets in a manner comparable to that of the corresponding glucoside. These analogs are designed to mimic or modify the biological activity of natural glucosides, potentially enhancing therapeutic effects or altering metabolic pathways in medical or industrial applications.

As used herein, “more than” refers to strictly greater than X. That is, the recitation that A is more than B indicates A>B.

As used herein, “less than” refers to be strictly below X. That is, the recitation that A is less than B indicates A<B.

As used herein, “Log P” refers to the concentration of analyte in organic phase/the concentration of analyte molecule in water phase. When the Log P is higher, the stronger of the lipophilicity of the analyte and the higher the degree of absorption by cell. An increase in Log P of the analyte is usually accompanied by a decrease in solubility in water, which represents rise of hydrophobicity of the analyte.

The present disclosure discloses a SGLT2 inhibitor derivative, comprising a backbone structure and a hydrophobic functional group, in which the backbone structure comprises a glucoside or a glucoside analog, in which the glucoside or the glucoside analog is bonded to the hydrophobic functional group via a covalent bond. By adding the hydrophobic functional group to the backbone structure, the SGLT2 inhibitor derivative has enhanced hydrophobicity, making it suitable for long-term treatment.

In some embodiments, the backbone structure is the glucoside, comprising a structure as below:

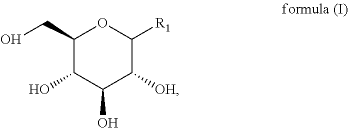

in which R₁ represents an unsubstituted aryl group, a substituted aryl group, an arylalkyl group, an arylalkylenearyl group, or a heteroaryl group, in which a substituent of the substituted aryl group comprises C₁-C₆ alkyl (such as C1 alkyl, C2 alkyl, C3 alkyl, C4 alkyl, C5 alkyl, or C6 alkyl), halo (such as, F, Cl, Br, I, or the like), alkoxy, alkenyl or heteroaryl. In some embodiments, the glucoside comprises Velagliflozin, Dapagliflozin, Canagliflozin, Empagliflozin, Tofogliflozin, Ipragliflozin, Atigliflozin, Bexagliflozin or Janagliflozin.

In some embodiments, the backbone structure is the glucoside analog, in which the glucoside analog comprises a first glucoside analog, a second glucoside analog or a third glucoside analog, in which the first glucoside analog represents oxygen-containing bridge skeleton formed in glucose six-membered ring of the glucoside, the second glucoside analog represents that oxygen atom in the glucose six-membered ring of the glucoside is replaced by sulfur, and the third glucoside analog represents carbon 6 in the glucoside is replaced by a methylthio group.

In some embodiments, the first glucoside analog comprises a structure as below:

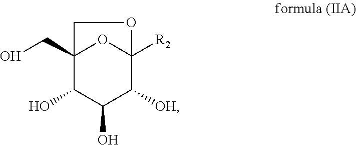

in which R₂ (the scope is similar to that of R₁) represents an unsubstituted aryl group, a substituted aryl group, an arylalkyl group, an arylalkylenearyl group, or a heteroaryl group, in which a substituent of the substituted aryl group comprises C₁-C₆ alkyl, halo, alkoxy, alkenyl or heteroaryl. In some embodiments, the first glucoside analog comprises Ertugliflozin and Henagliflozin.

In some embodiments, the second glucoside analog comprises a structure as below:

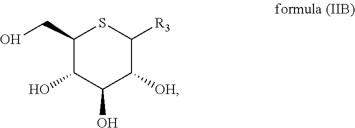

in which R₃ (the scope is similar to that of R₁) represents an unsubstituted aryl group, a substituted aryl group, an arylalkyl group, an arylalkylenearyl group, or a heteroaryl group, in which a substituent of the substituted aryl group comprises C₁-C₆ alkyl, halo, alkoxy, alkenyl or heteroaryl. In some embodiments, the second glucoside analog comprises Luseogliflozin.

In some embodiments, the third glucoside analog comprises a structure as below:

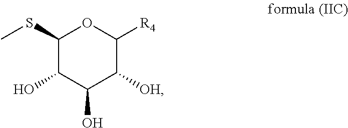

in which R₄ (the scope is similar to that of R₁) represents an unsubstituted aryl group, a substituted aryl group, an arylalkyl group, an arylalkylenearyl group, or a heteroaryl group, in which a substituent of the substituted aryl group comprises C₁-C₆ alkyl, halo, alkoxy, alkenyl or heteroaryl. In some embodiments, the third glucoside analog comprises Sotagliflozin.

In some embodiments, when the backbone structure is the glucoside, the SGLT2 inhibitor derivative has a structure of formula (III):

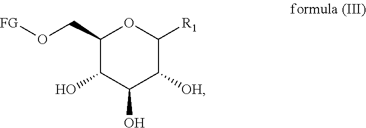

in which when the backbone structure is the glucoside analog, the SGLT2 inhibitor derivative has a structure of formula (IVA), formula (IVB) or formula (IVC):

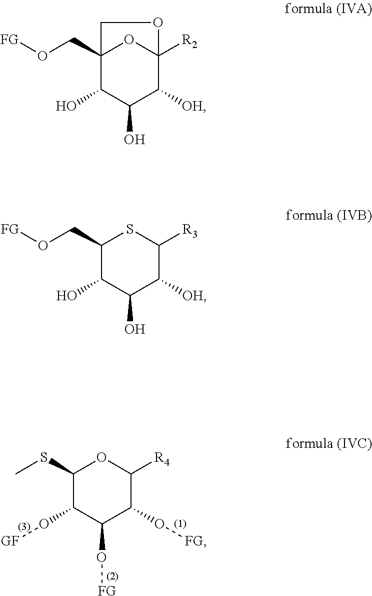

in which R₁, R₂, R₃, R₄ represents an unsubstituted aryl group, a substituted aryl group, an arylalkyl group, an arylalkylenearyl group, or a heteroaryl group, respectively, a substituent of the substituted aryl group comprises C₁-C₆ alkyl, halo, alkoxy, alkenyl or heteroaryl, and FG represents the hydrophobic functional group is bonded to O at position (1), position (2), position (3) or a combination thereof. In some embodiments, one hydrophobic functional group is bonded to position (1), position (2) or position (3). In some embodiments, three hydrophobic functional groups are bonded to position (1), position (2) and position (3), respectively.

In some embodiments, the hydrophobic functional group comprises an aliphatic group, an aromatic ring group or a combination thereof, in which the aliphatic group has at least 6 carbon atoms (such as 6, 7, 8, 9, 10, 11, 12, 13, 14, 15, 16, 17, 18, 19, 20, 21, 22, 23, 24, 25, 26, 27, 28, 29, 30 carbon atoms). It can be understood that the design of a minimum of six carbon atoms significantly enhances hydrophobic properties, and as the number of carbon atoms increases, the hydrophobic property of SGLT2 inhibitor derivative is enhanced accordingly.

In some embodiments, the aliphatic group comprises hexyl, octyl, decyl, dodecyl, tetradecyl, hexadecyl, octadecyl, icosyl, docosyl, tetracosyl, caproyl, capryloyl, decanoyl, dodecanoyl, tetradecanoyl, hexadecanoyl, octadecanoyl, icosanoyl, docosanoyl, tetracosanoyl or a combination thereof. In some embodiments, the aromatic ring group comprises cinnamoyl, phenylacetyl, indanyl, salicyl, anthranilyl, benzoyl or a combination thereof. In some embodiments, the SGLT2 inhibitor derivative is carbonate ester derivative (such as hexyl carbonate ester derivative), fatty acid ester derivative (such as hexyl ester derivative, hexadecyl ester derivative or docosyl ester derivative), phosphodiester derivative, aromatic ester derivative (such as 2,3-dihydro-1H-indene-5-carboxylate ester derivative) or carbamate derivative. In some embodiments, an illustrative trend of increasing hydrophobicity is observed from hexyl ester derivative, 2,3-dihydro-1H-indene-5-carboxylate ester derivative, hexyl carbonate ester derivative, hexadecyl ester derivative, and docosyl ester derivative.

In some embodiments, the hydrophobic functional group is bonded at the oxygen atom of C2, C3, C4, or C6 of the glucoside or the glucoside analog. For example, the hydrophobic functional group is bonded to the OH group of the glucoside or the glucoside analog. In some embodiments, the covalent bond comprises an ester bond, an acetal bond or a ketal bond. In some embodiments, the ester bond is a carbonate ester bond, a fatty acid ester bond, a phosphodiester bond, an aromatic ester bond, or a carbamate bond. It's noted that when the ester bond is formed in the SGLT2 inhibitor derivative, the resulting SGLT2 inhibitor derivative functions as a prodrug by masking the parent SGLT2 inhibitor (the glucoside or the glucoside analog). Under physiological conditions, the hydrolysis of ester linkages occurs, thereby enabling delayed release of the active SGLT2 inhibitor. Such modifications may be carried out using conventional functional group transformation techniques well known to those skilled in the art, including, but not limited to, esterification, carbonate formation, carbamate formation, acetal formation, or ketal formation.

In some embodiments, the SGLT2 inhibitor derivative, formed by the combination of the glucoside and the hydrophobic functional group or the combination of the glucoside analog and the hydrophobic functional group, demonstrates enhanced hydrophobicity compared to the original SGLT2 inhibitors comprising the glucoside or the glucoside analog. In some embodiments, the SGLT2 inhibitor derivative has a hydrophobic constant of more than 3, such as 3.2, 3.5, 4.0, 4.5, 5.0, 5.5, 6.0, 6.5, 7.0, 7.5, 8.0, 8.5, 9.0, 10.5, 11.0, 11.5, 12, 12.5, 13.0, 13.5, 14, or a value within the interval defined between any two of the foregoing numerical values. In some embodiments, the hydrophobic constant is calculated by X Log P3 or measured by a distribution coefficient test of n-octanol-water system. In some embodiments, the SGLT2 inhibitor derivative is very slightly soluble (<0.1 mg/mL) in the phosphate buffer of pH 2, pH 4, pH 6, pH 8, pH 10 or pH 12. For example, a solubility of the SGLT2 inhibitor derivative is more than 0 and less than 0.1 mg/ml in pH 6 phosphate buffer.

### 1. Hydrophobicity Prediction of SGLT2 Inhibitor Derivatives

To elucidate how to predict hydrophobicity of SGLT2 inhibitor derivatives more precisely, two Log P prediction methods, C Log P (calculated by Chem Draw software) and X Log P3 (J. Chem. Inf. Model. 2007, 47, 2140-2148; website: https://www.swissadme.ch/), were selected to predict Log P of Dapagliflozin, and the predicted values (C Log P and x Log 3) were compared with the actual value of Log P (counted by n-octanol-water system experiment). The result was represented as Table 1-1.

TABLE 1-1
Log P Values between Different Prediction Models

| Actual Value of LogP | CLogP | XLogP3 |
|---|---|---|
| 2.27 | 3.36 | 2.35 |

As shown in Table 1-1, since the X log P3 calculation model was closer to the actual value, X Log P3 was used as the main prediction tool in the following, and its value was defined as the hydrophobic constant.

Furthermore, hydrophobic constant of several types of SGLT2 inhibitor derivatives was predicted by X Log P3 and conducted in Table 1-2.

TABLE 1-2
Predicted Hydrophobic Constant of SGLT2 Inhibitor Derivative

| | Hydrophobic Constant (XLogP3) | | | | |
|---|---|---|---|---|---|
| | Hexyl | | | | |
| | Carbonate | Aromatic | Hexyl | Hexadecyl | Docosyl |
| | Ester | Ester | Ester | Ester | Ester |
| SGLT2 Inhibitor | Derivative | Derivative | Derivative | derivative | Derivative |
| Velagliflozin | 4.77 | 4.53 | 3.94 | 9.36 | 12.70 |
| Dapagliflozin | 5.11 | 4.87 | 4.28 | 9.70 | 12.95 |
| Canagliflozin | 5.99 | 5.75 | 5.16 | 10.58 | 13.92 |
| Empagliflozin | 4.79 | 4.55 | 4.00 | 9.38 | 12.77 |
| Tofogliflozin | 4.28 | 4.04 | 3.45 | 8.87 | 12.21 |
| Ipragliflozin | 5.30 | 5.06 | 4.47 | 9.89 | 13.23 |
| Atigliflozin | 4.13 | 3.89 | 3.30 | 8.72 | 12.06 |
| Bexagliflozin | 5.16 | 4.92 | 4.32 | 9.75 | 13.08 |
| Janagliflozin | 6.13 | 5.89 | 5.30 | 10.72 | 14.06 |
| Henagliflozin | 4.58 | 4.34 | 3.75 | 9.17 | 12.51 |
| Ertugliflozin | 4.48 | 4.24 | 3.65 | 9.07 | 12.41 |
| Luseogliflozin | 5.62 | 5.38 | 4.79 | 10.21 | 13.55 |

Table 1-2 presented several SGLT2 inhibitor derivatives and their calculated hydrophobic constant values calculated using the X Log P3 model. It was evident that the X Log P3 values of the SGLT2 inhibitor derivatives were all no less than 3 (e.g., the aromatic ester derivative of Atigliflozin was 3.89; the hexyl ester derivative of Tofogliflozin was 3.45), thereby validating that the addition of the hydrophobic functional group enhanced SGLT2 inhibitor derivative hydrophobicity.

For example, it was represented that through the synthetic design of dapagliflozin derivatives, the hydrophobic constant value increased to no less than 4. For example, the hexyl carbonate ester of dapagliflozin had a hydrophobic constant of 5.11, which represented an increase of 2.76 compared with the X Log P3 value of dapagliflozin (2.35) itself. The same trend was observed in other SGLT2 inhibitor derivatives modified with hydrophobic functional groups. That is, compared with the original SGLT2 inhibitors prior to synthesis, the SGLT2 inhibitor derivatives exhibited enhanced hydrophobic constants in the prediction model.

### 2. Synthesis Products of SGLT2 Inhibitor Derivatives

### Example 1: Dapagliflozin Palmitate (Hexadecyl) Ester Derivative

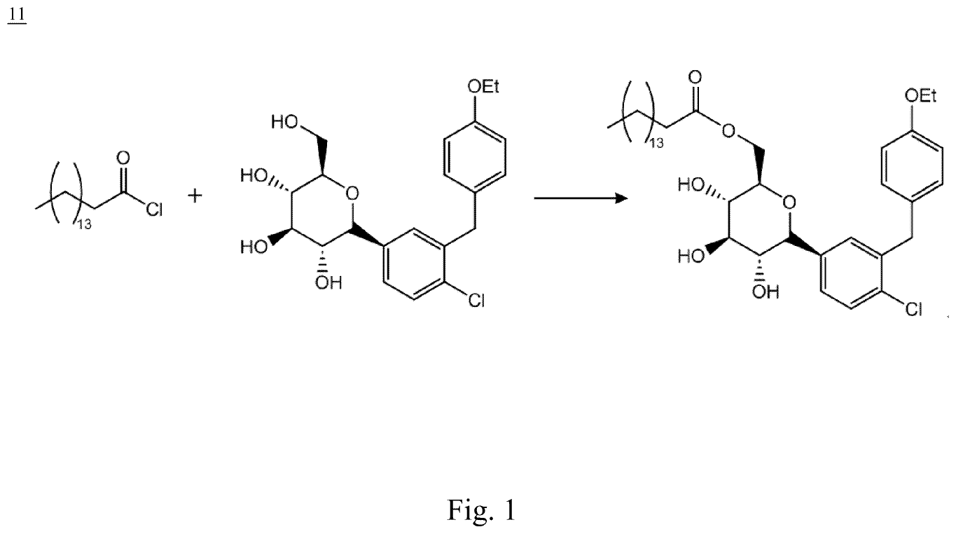

As shown in FIG. 1 (synthesis diagram 11), the synthesis method of dapagliflozin hexadecyl ester derivative was as follows.

70 g dapagliflozin and 51.97 g triethylamine was dissolved in 1.4 L dichloromethane under 10° C. conditions. After complete dissolution, a solution was formed, and 61.18 g palmitoyl chloride was added into the solution mentioned before. After stirring at 0° C.-5° C. and 1200 rpm for 2-4 hours, the reaction was terminated with half the volume of the organic phase of 10% sodium chloride solution to quench residual acyl chloride. The organic solvent (dichloromethane) was removed under reduced pressure. The product was then purified by column chromatography and dried in vacuum. The yield of the obtained sample was 60% and the appearance was a light-yellow solid.

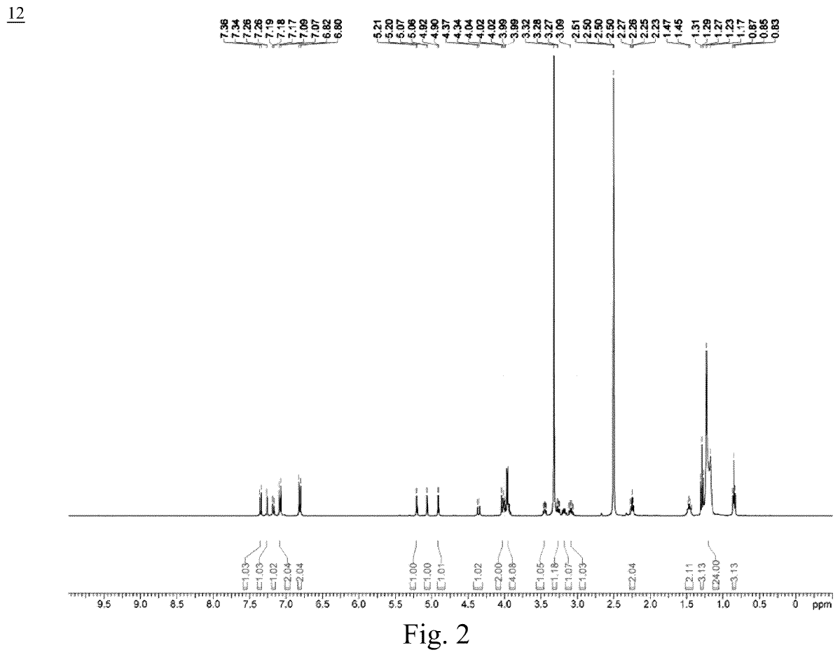

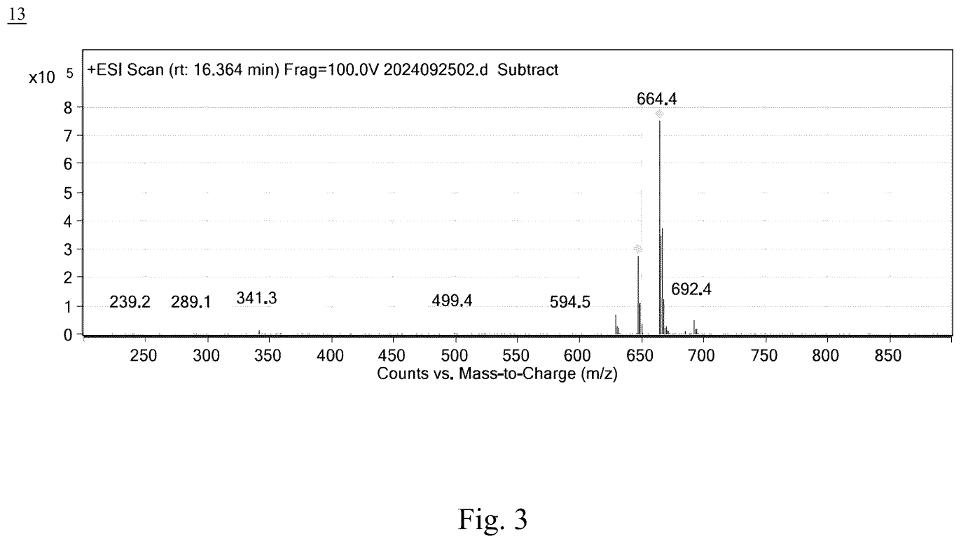

¹H-NMR (proton nuclear magnetic resonance) spectrum and LC-MS (liquid chromatography-mass spectrometry) spectrum of the obtained sample were represented in FIG. 2 (NMR spectrum 12) and FIG. 3 (LC-MS spectrum 13), respectively, in which ¹H-NMR spectrum represented ¹H-NMR (DMSO-d6, 400 MHz, 25° C., zg30 pulse seq.) δ ppm: 0.83-0.87 (triplet, 3H, CH₃, methyl), 1.17-1.23 (24H, —C₁₂H₂₄—, twelve methylene), 1.27-1.31 (triplet, 3H, CH₃, methyl), 1.45-1.47 (quintet, 2H, CH₂, methylene), 2.23-2.27 (triplet, 2H, CH₂, methylene), 3.09 (1H, CH, tetrahydropyran), 3.27 (1H, CH, tetrahydropyran), 3.28 (1H, CH, tetrahydropyran), 3.32 (1H, CH, tetrahydropyran), 3.90-3.97 (4H, two CH₂, methylene), 3.99-4.04 (2H, CH₂, methylene), 4.34-4.37 (1H, CH, tetrahydropyran), 4.90-4.92 (doublet, 1H, OH, alcohol), 5.06-5.07 (doublet, 1H, OH, alcohol), 5.20-5.21 (doublet, 1H, OH, alcohol), 6.80 (doublet, 1H, CH, benzyl), 6.82 (doublet, 1H, CH, benzyl), 7.07 (doublet, 1H, CH, benzyl), 7.09 (doublet, 1H, CH, benzyl), 7.17-7.19 (doublet, 1H, CH, benzyl), 7.26 (doublet, 1H, CH, benzyl) and 7.34-7.36 (doublet, 1H, CH, benzyl). Hydrophobic constant was predicted using X Log P3 model methods and X Log P3 was 9.70, which was 7.35 higher than the unmodified dapagliflozin whose X Log P3 was 2.35. The pKa was 13.1 (ACD Labs software). LC-MS was analyzed by the condition of (ESI⁺, 100V): m/z=664.4 [M+NH4]+.

### Example 2: Dapagliflozin Hexyl Carbonate Ester Derivative

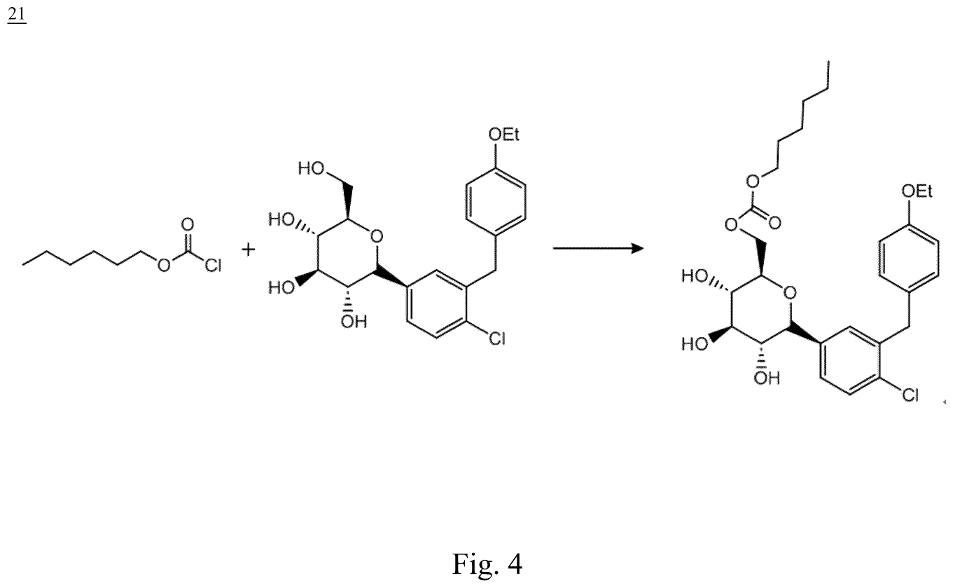

As shown in FIG. 4 (synthesis diagram 21), the synthesis method of dapagliflozin hexyl carbonate ester derivative was as follows.

180 g dapagliflozin and 133.6 g triethylamine were dissolved in 3.6 L dichloromethane under 10° C. conditions. After complete dissolution, 94.2 g hexyl chloroformate was added and stirred at 0° C.-5° C. and 1200 rpm for 2-4 hours, the reaction was terminated with half the volume of the organic phase of 10% sodium chloride solution to quench residual acyl chloride. The organic solvent (dichloromethane) was removed under reduced pressure. The product was then purified by column chromatography and dried in vacuum. The yield of the obtained sample was 70% and the appearance was a light-yellow solid.

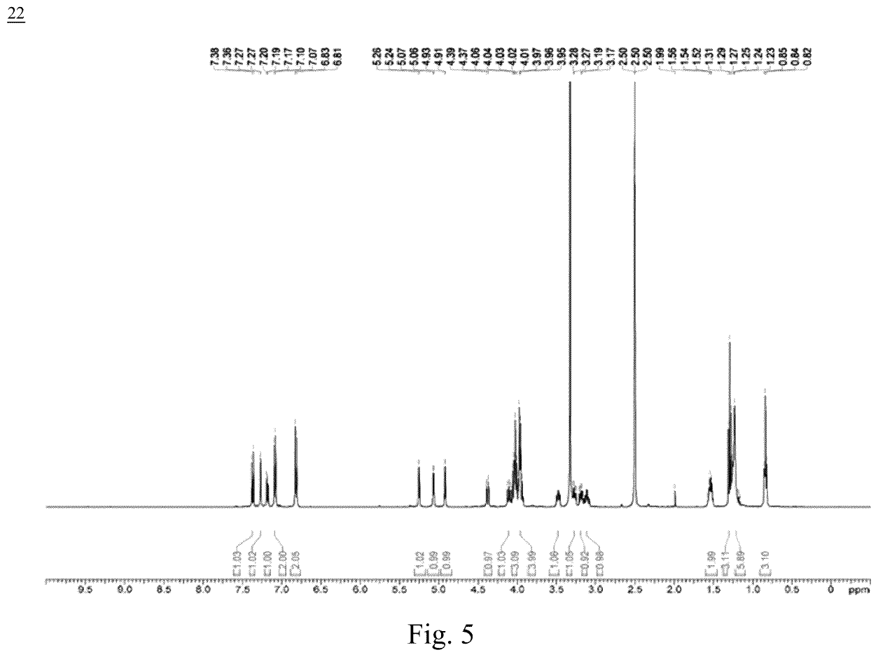

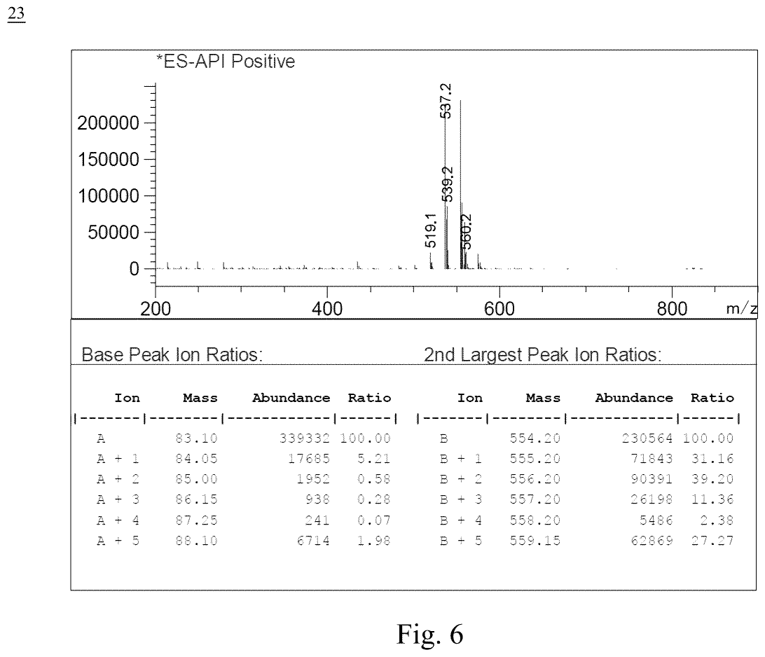

¹H-NMR spectrum and LC-MS spectrum of the obtained sample were represented in FIG. 5 (NMR spectrum 22) and FIG. 6 (LC-MS spectrum 23), respectively, in which ¹H-NMR spectrum represented (DMSO-d6, 400 MHz, 25° C., zg30 pulse seq.) δ ppm: 0.82-0.85 (triplet, 3H, CH₃, methyl), 1.23-1.25 (6H, —C₃H₆—, three methylene), 1.27-1.31 (triplet, 3H, CH₃, methyl), 1.52-1.56 (quintet, 2H, CH₂, methylene), 3.09-3.15 (1H, CH, tetrahydropyran), 3.17-3.23 (1H, CH, tetrahydropyran), 3.28 (1H, CH, tetrahydropyran), 3.45-3.51 (1H, CH, tetrahydropyran), 3.99 (4H, two CH₂, methylene), 4.00-4.05 (2H, CH₂, methylene), 4.05-4.09 (2H, CH₂, methylene), 4.35-4.39 (1H, CH, tetrahydropyran), 4.91-4.93 (doublet, 1H, OH, alcohol), 5.06-5.07 (doublet, 1H, OH, alcohol), 5.24-5.26 (doublet, 1H, OH, alcohol), 6.81 (doublet, 1H, CH, benzyl), 6.83 (doublet, 1H, CH, benzyl), 7.07 (doublet, 1H, CH, benzyl), 7.10 (doublet, 1H, CH, benzyl), 7.17-7.20 (doublet, 1H, CH, benzyl), 7.27 (doublet, 1H, CH, benzyl) and 7.36-7.38 (doublet, 1H, CH, benzyl). Hydrophobic constant was predicted using X Log P3 model methods and X Log P3 was 5.11, which was 2.76 higher than the unmodified dapagliflozin whose X Log P3 was 2.35. The pKa was 13.1 (ACD Labs software). LC-MS was analyzed by the condition of (ESI⁺, 100V): m/z=537.2 [M+H]+.

### Example 3: Dapagliflozin Aromatic Ester Derivative

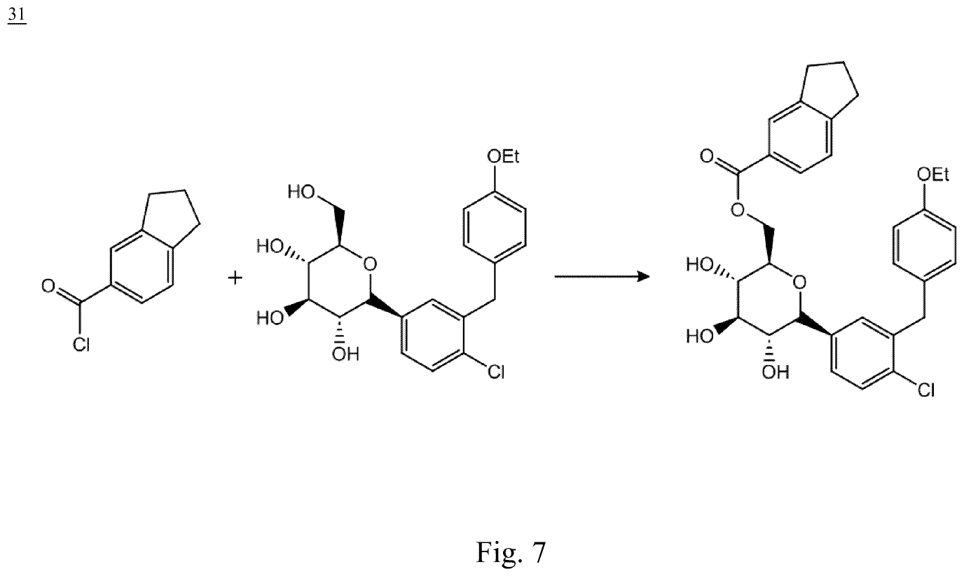

As shown in FIG. 7 (synthesis diagram 31), the synthesis method of dapagliflozin aromatic ester derivative was as follows.

200 g dapagliflozin and 148.0 g triethylamine was dissolved in 4.0 L dichloromethane under 10° C. conditions. After complete dissolution, a solution was formed, and 114.8 g 2,3-dihydro-1H-indene-5-carbonyl chloride was added and stirred at 0° C.-5° C. and 1200 rpm for 2-4 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum. The yield of the obtained sample was 70% and the appearance was a yellow solid.

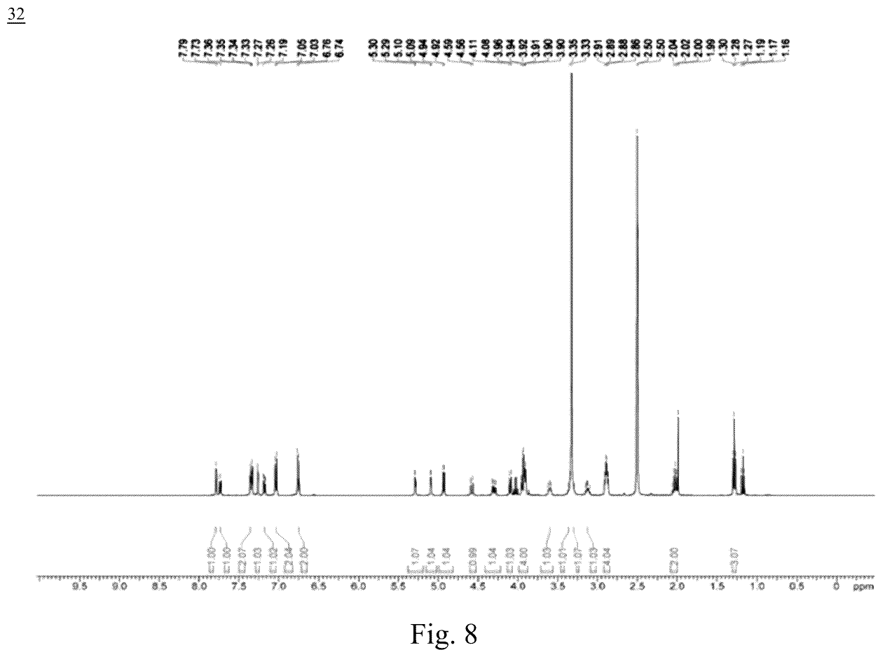

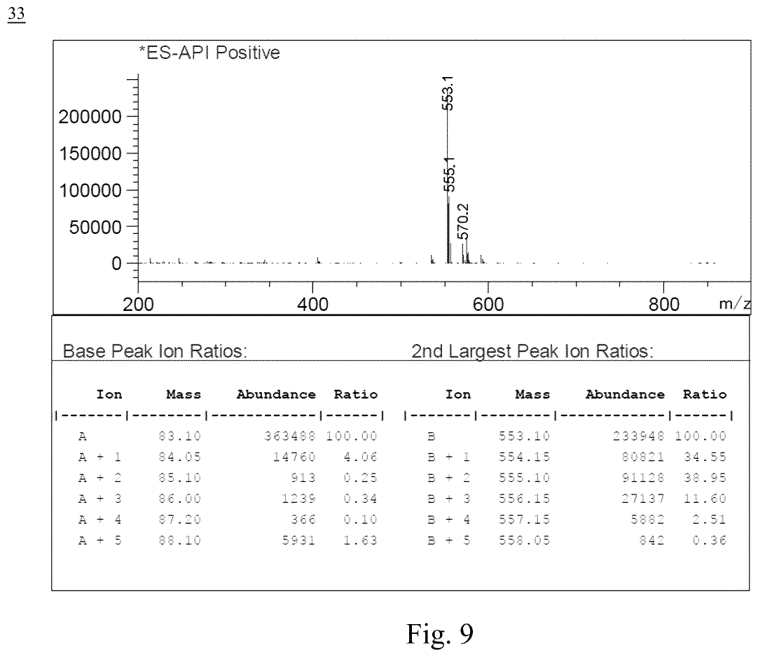

¹H-NMR spectrum and LC-MS spectrum of the obtained sample were represented in FIG. 8 (NMR spectrum 32) and FIG. 9 (LC-MS spectrum 33), respectively, in which ¹H-NMR spectrum represented ¹H-NMR (DMSO-d6, 400 MHz, 25° C., zg30 pulse seq.) δ ppm: 1.27-1.30 (triplet, 3H, CH₃, methyl), 2.00-2.04 (quintet, 2H, CH₂, indan), 2.86-2.91 (triplet, 4H, two CH₂, indan), 3.08-3.17 (1H, CH, tetrahydropyran), 3.25-3.31 (1H, CH, tetrahydropyran), 3.36-3.41 (1H, CH, tetrahydropyran), 3.56-3.64 (1H, CH, tetrahydropyran), 3.85-3.98 (4H, two CH₂, methylene), 4.06-4.14 (1H, CH, tetrahydropyran), 4.25-4.35 (1H, CH, tetrahydropyran), 4.92-4.94 (doublet, 1H, OH, alcohol), 5.09-5.10 (doublet, 1H, OH, alcohol), 5.29-5.30 (doublet, 1H, OH, alcohol), 6.74 (doublet, 1H, CH, benzyl), 6.76 (doublet, 1H, CH, benzyl), 7.03 (doublet, 1H, CH, benzyl), 7.05 (doublet, 1H, CH, benzyl), 7.19 (doublet, 1H, CH, benzyl), 7.26 (doublet, 1H, CH, benzyl), 7.33 (doublet, 1H, CH, benzyl), 7.34-7.36 (triplet, 1H, CH, indan) and 7.71-7.85 (2H, two CH, indan). Hydrophobic constant was predicted using X Log P3 model methods and X Log P3 was 4.87, which was 2.52 higher than the unmodified dapagliflozin whose X Log P3 was 2.35. The pKa was 13.1 (ACD Labs software). LC-MS was analyzed by the condition of (ESI⁺, 100V): m/z=553.1 [M+H]+.

### Example 4: Empagliflozin Palmitate (Hexadecyl) Ester Derivative

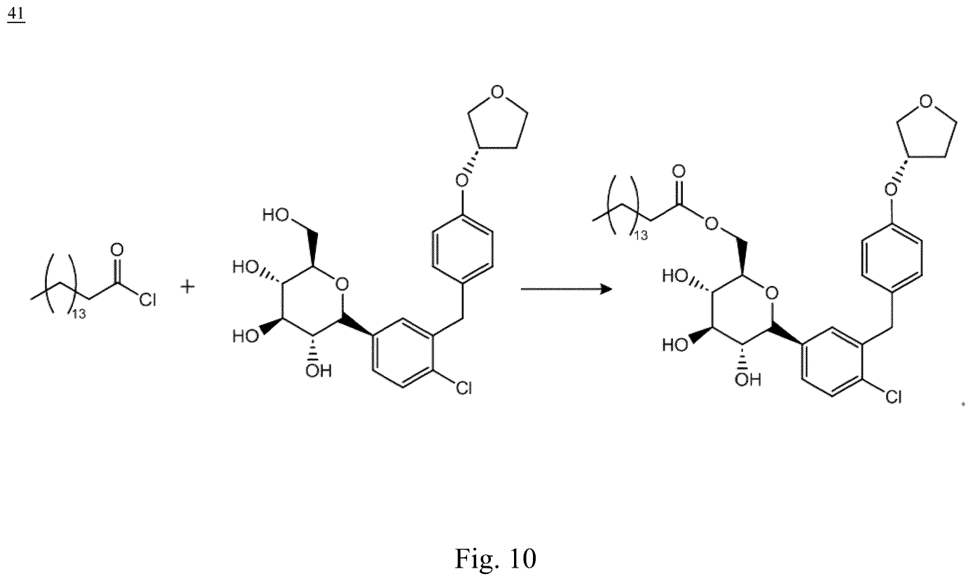

As shown in FIG. 10 (synthesis diagram 41), the synthesis method of empagliflozin hexadecyl Ester Derivative was as follows.

190 g empagliflozin was dissolved in 190 mL pyridine, 2850 ml tetrahydrofuran and 2850 mL dichloromethane under 10° C. conditions. After complete dissolution, a solution was formed, and 139 g palmitoyl chloride was added into the solution mentioned before. After stirring at 10° C. and 1200 rpm for 2 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum. The yield of the obtained sample was 35% and the appearance was a white solid.

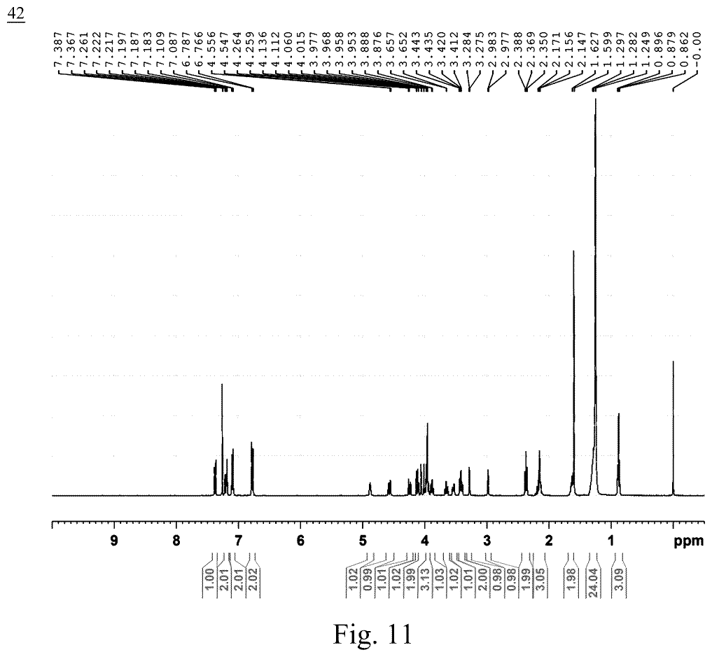

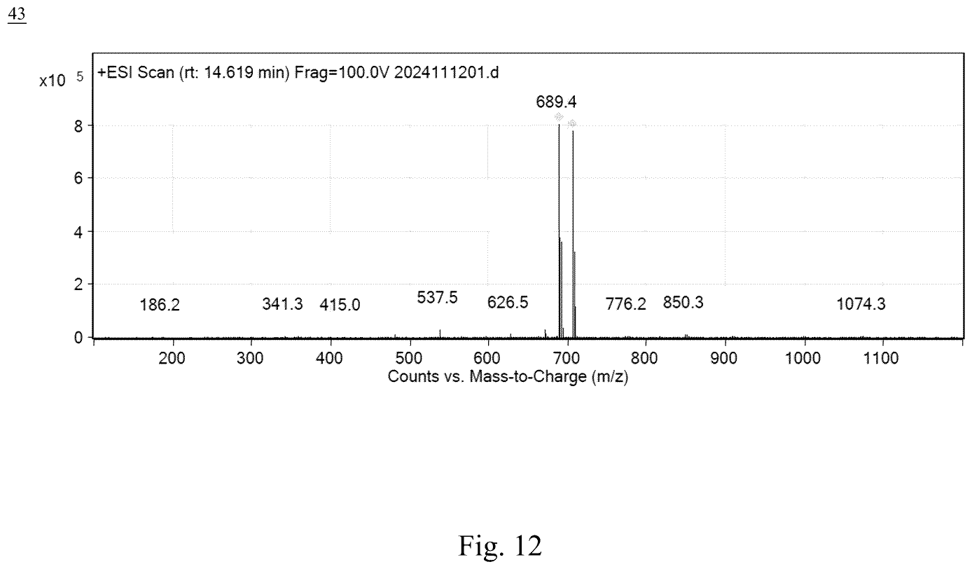

¹H-NMR spectrum and LC-MS spectrum of the obtained sample were represented in FIG. 11 (NMR spectrum 42) and FIG. 12 (LC-MS spectrum 43), respectively, in which ¹H-NMR spectrum represented ¹H-NMR (CDCl₃, 400 MHz, 25° C., zg30 pulse seq.) δ ppm: 0.86-0.90 (triplet, 3H, CH₃, methyl), 1.23-1.30 (24H, —C₁₂H₂₄—, twelve methylene), 1.60-1.62 (quintet, 2H, CH₂, methylene), 2.98 (1H, CH, tetrahydropyran), 3.28 (1H, CH, tetrahydropyran), 3.42 (2H, CH₂, tetrahydropyran), 3.55 (1H, CH, tetrahydropyran), 3.65 (1H, CH, tetrahydropyran), 3.85-3.99 (2H, CH₂, methylene; 2H, CH₂, tetrahydropyran), 3.99-4.11 (2H, CH₂, methylene), 4.11-4.16 (1H, CH, tetrahydropyran), 4.20-4.29 (doublet, 1H, OH, alcohol), 4.51-4.62 (doublet, 1H, OH, alcohol), 4.83-4.94 (doublet, 1H, OH, alcohol), 6.76-6.79 (doublet, 2H, two CH, benzyl), 7.08-7.11 (doublet, 2H, two CH, benzyl), 7.18-7.26 (doublet, 2H, two CH, benzyl) and 7.36-7.39 (doublet, 1H, CH, benzyl). Hydrophobic constant was predicted using X Log P3 model methods and X Log P3 was 9.38, which was 7.35 higher than the unmodified empagliflozin whose X Log P3 was 2.03. The pKa was 12.9 (ChemDraw). LC-MS was analyzed by the condition of (ESI⁺, 100V): m/z=689.4 [M+H]+.

### Example 5: Empagliflozin Hexyl Carbonate Ester Derivative

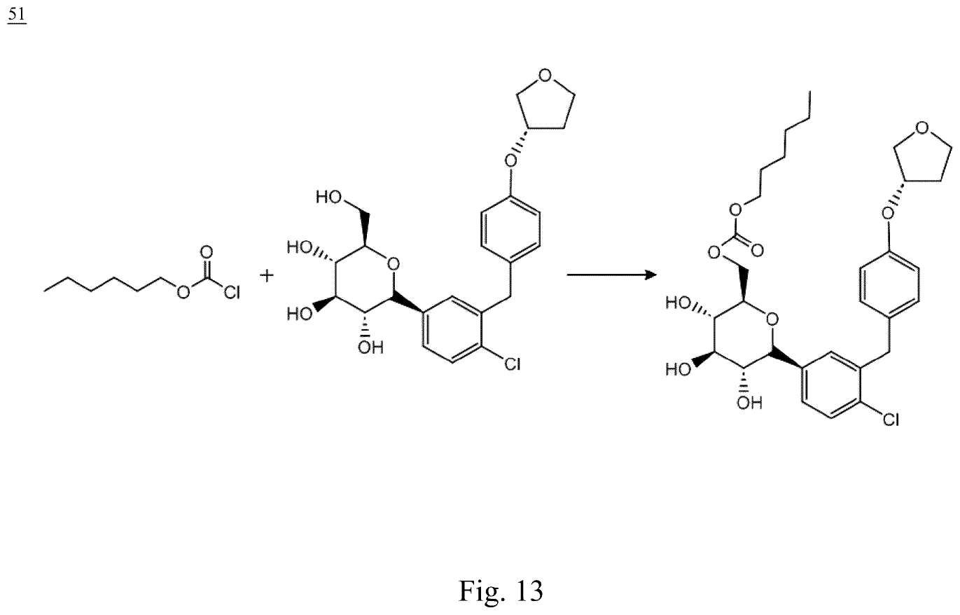

As shown in FIG. 13 (synthesis diagram 51), the synthesis method of empagliflozin hexyl carbonate ester derivative was as follows.

110 g empagliflozin was dissolved in 110 mL pyridine, 1650 mL tetrahydrofuran and 1650 mL dichloromethane under 10° C. conditions. After complete dissolution, 52.21 g hexyl chloroformate and 2.98 g DMAP was added and stirred at 10° C. and 1200 rpm for 2 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum. The yield of the obtained sample was 36% and the appearance was a white solid.

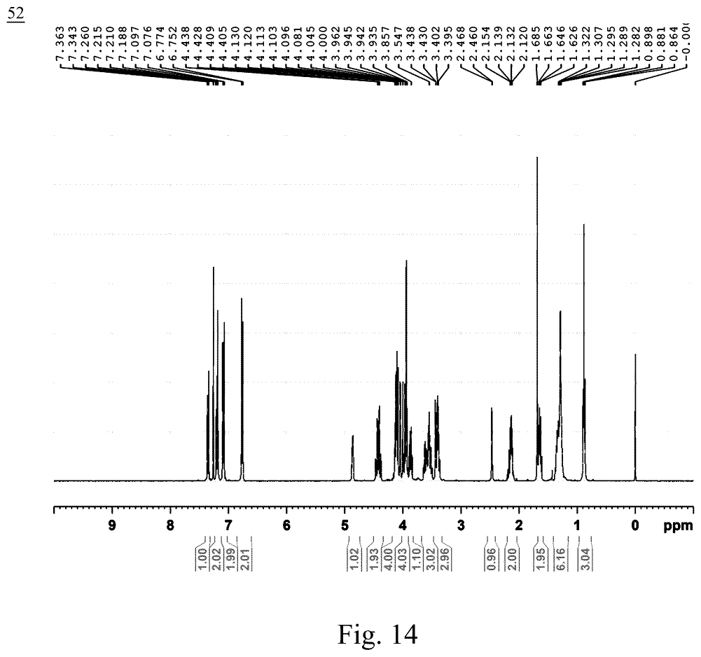

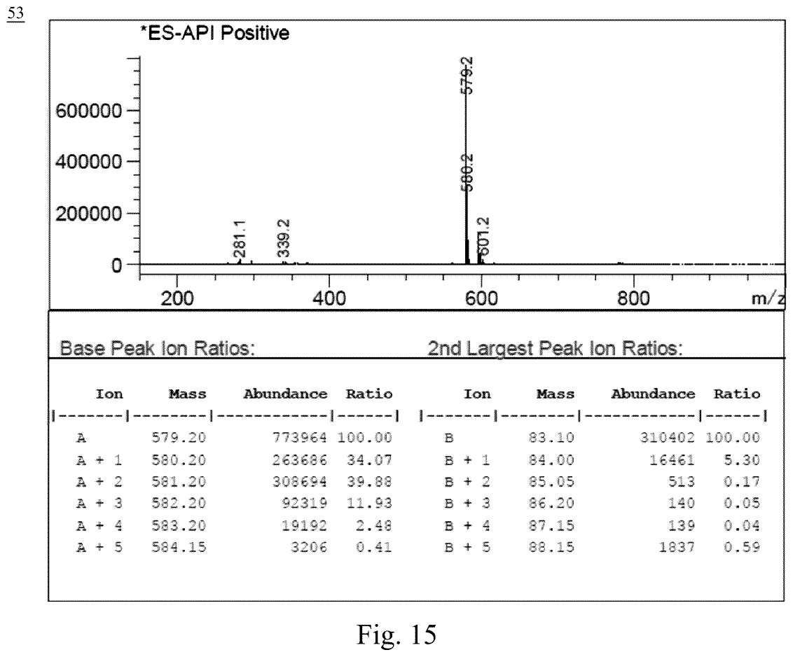

¹H-NMR spectrum and LC-MS spectrum of the obtained sample were represented in FIG. 14 (NMR spectrum 52) and FIG. 15 (LC-MS spectrum 53), respectively, in which ¹H-NMR spectrum represented ¹H-NMR (CDCl₃, 400 MHz, 25° C., zg30 pulse seq.) δ ppm: 0.86-0.90 (triplet, 3H, CH₃, methyl), 1.17-1.42 (6H, —C₃H₆—, three methylene), 1.62-1.69 (quintet, 2H, CH₂, methylene), 2.12-2.14 (quartet, 2H, CH₂, tetrahydropyran), 4.83-4.94 (doublet, 1H, OH, alcohol), 6.75-6.77 (doublet, 2H, two CH, benzyl), 7.07-7.09 (doublet, 2H, two CH, benzyl), 7.18-7.26 (doublet, 2H, two CH, benzyl) and 7.34-7.36 (doublet, 1H, CH, benzyl). Hydrophobic constant was predicted using X Log P3 model methods and X Log P3 was 4.79, which was 2.76 higher than the unmodified empagliflozin whose X Log P3 was 2.03. The pKa was 12.9 (ChemDraw). LC-MS was analyzed by the condition of LC-MS analyze (ESI⁺, 100V): m/z=579.2 [M+H]+.

### Example 6: Empagliflozin Aromatic Ester Derivative

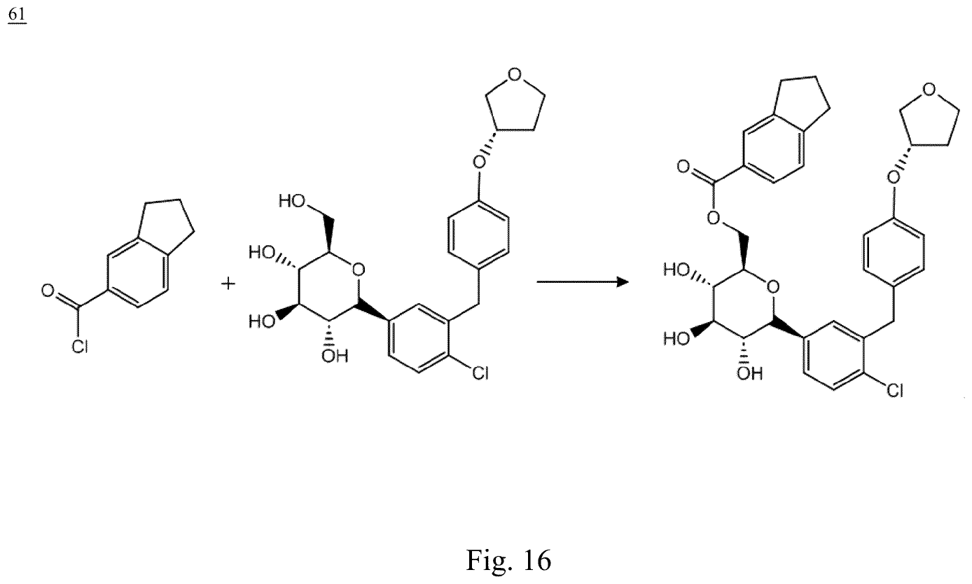

As shown in FIG. 16 (synthesis diagram 61), the synthesis method of empagliflozin aromatic ester derivative was as follows.

222.4 g empagliflozin was dissolved in 222.4 mL pyridine and 3336 mL dichloromethane under 10° C. conditions. After complete dissolution, 89.1 g 2,3-dihydro-1H-indene-5-carbonyl chloride was added and stirred at 0° C.-5° C. and 1200 rpm for 2-4 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum. The yield of the obtained sample was 41% and the appearance was a yellow solid.

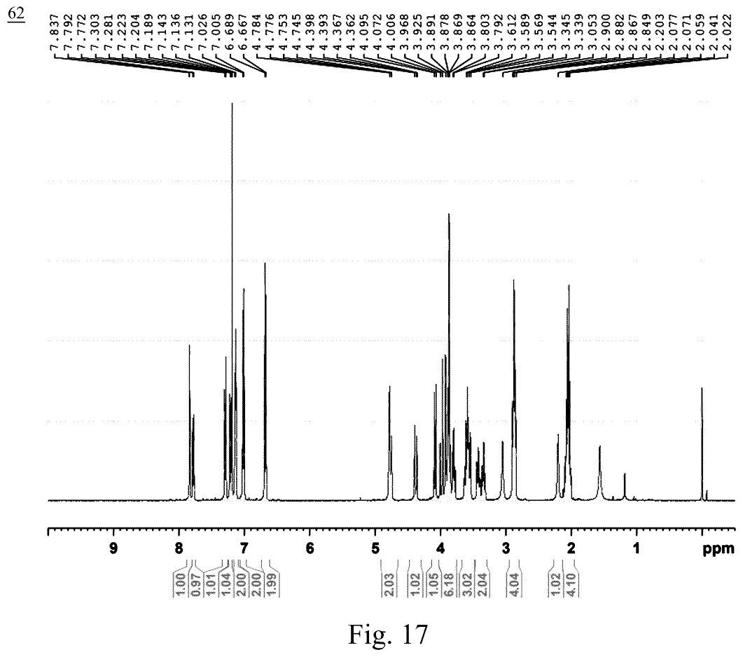

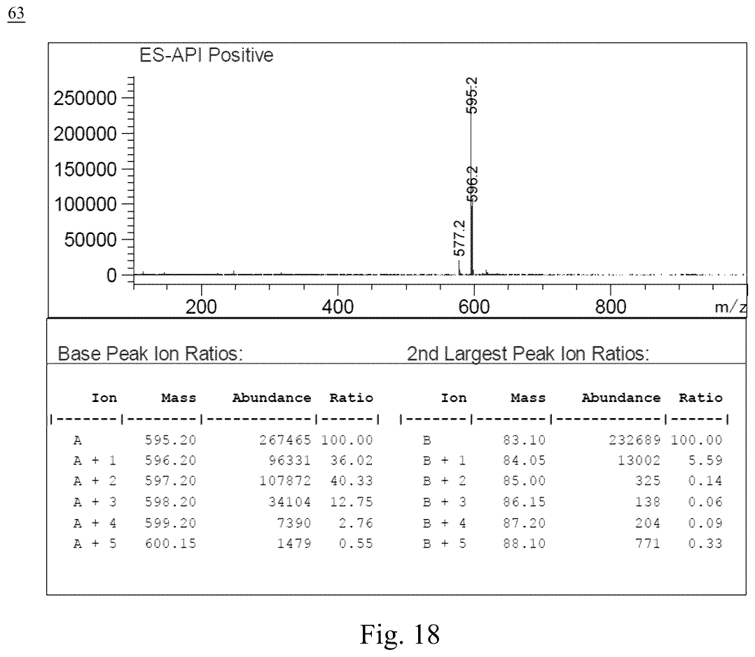

¹H-NMR spectrum and LC-MS spectrum of the obtained sample were represented in FIG. 17 (NMR spectrum 62) and FIG. 18 (LC-MS spectrum 63), respectively, in which ¹H-NMR spectrum represented ¹H-NMR (CDCl₃, 400 MHz, 25° C., zg30 pulse seq.) δ ppm: 1.95-2.15 (quartet, 2H, CH₂, tetrahydropyran; quintet, 2H, CH₂, indan), 2.80-2.95 (triplet, 4H, two CH₂, indan), 4.00 (singlet, 1H, OH, alcohol), 6.67-6.69 (doublet, 2H, two CH, benzyl), 7.00-7.03 (doublet, 2H, two CH, benzyl), 7.13-7.14 (doublet, 2H, two CH, benzyl), 7.21-7.24 (triplet, 1H, CH, indan), 7.28-7.31 (doublet, 1H, CH, benzyl) and 7.77-7.85 (2H, two CH, indan). Hydrophobic constant was predicted using X Log P3 model methods and X Log P3 was 4.55, which was 2.52 higher than the unmodified empagliflozin whose X Log P3 was 2.03. The pKa was 12.9 (ChemDraw). LC-MS was analyzed by the condition of (ESI⁺, 100V): m/z=595.2 [M+H]+.

### Example 7: Bexagliflozin Palmitate (Hexadecyl) Ester Derivative

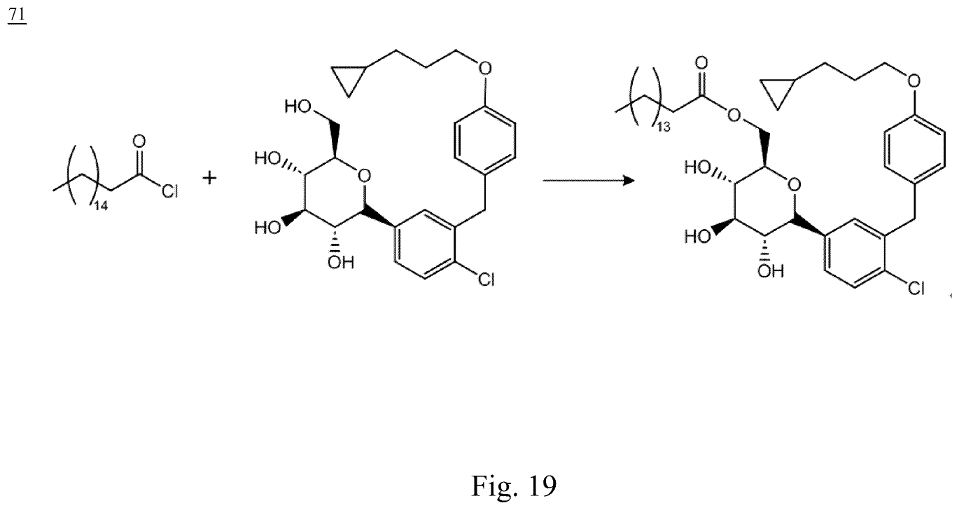

As shown in FIG. 19 (synthesis diagram 71), the synthesis method of bexagliflozin hexadecyl ester derivative was as follows.

180 g bexagliflozin was dissolved in 180 mL pyridine, 1800 mL tetrahydrofuran and 1800 mL dichloromethane under 10° C. conditions. After complete dissolution, 138.34 g palmitoyl chloride was added and stirred at 10° C. and 1200 rpm for 18-20 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum. The yield of the obtained sample was 31% and the appearance was a nearly-white solid.

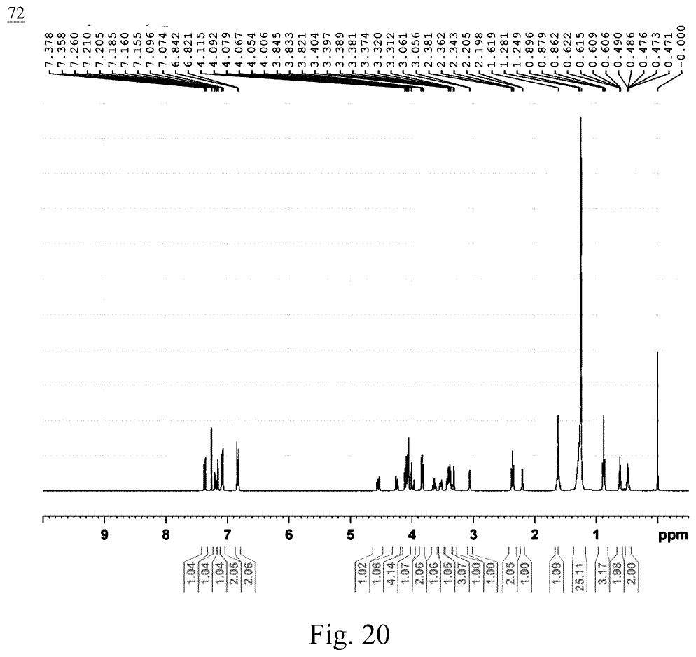

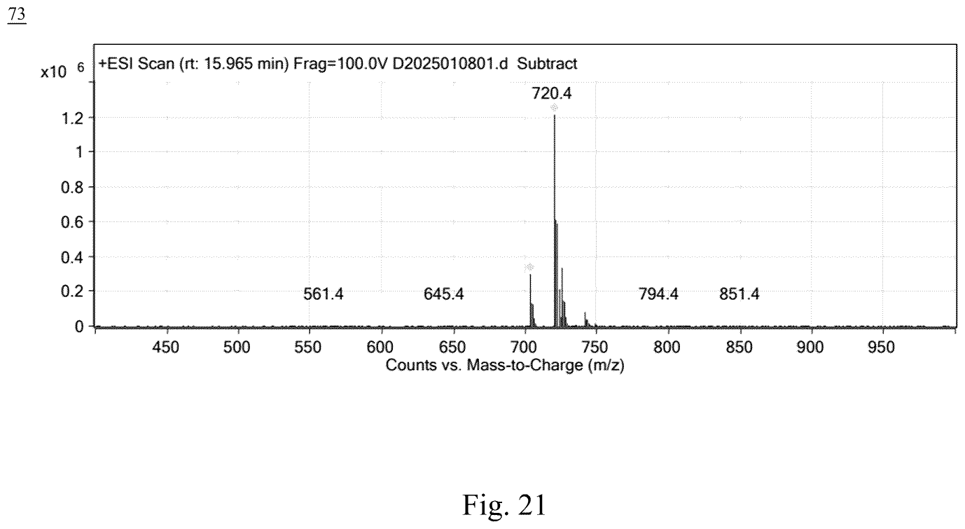

¹H-NMR spectrum and LC-MS spectrum of the obtained sample were represented in FIG. 20 (NMR spectrum 72) and FIG. 21 (LC-MS spectrum 73), respectively, in which ¹H-NMR spectrum represented ¹H-NMR (CDCl₃, 400 MHz, 25° C., zg30 pulse seq.) δ ppm: 0.47-0.49; 0.60-0.62 (4H, CH₂, cyclopropane), 0.86-0.90 (triplet, 3H, CH₃, methyl), 1.24-1.30; 1.60-1.62 (26H, —C₁₃H₂₆—, thirteen methylene), 2.34-2.38 (2H, CH₂, methylene), 6.82-6.84 (doublet, 2H, CH, benzyl), 7.07-7.10 (doublet, 2H, CH, benzyl), 7.17-7.20 (doublet, 1H, CH, benzyl), 7.27 (doublet, 1H, CH, benzyl) and 7.36-7.38 (doublet, 1H, CH, benzyl). Hydrophobic constant was predicted using X Log P3 model methods and X Log P3 was 9.75, which was 7.35 higher than the unmodified bexagliflozin whose X Log P3 was 2.40. The pKa was 18.86 (ChemDraw). LC-MS was analyzed by the condition of (ESI⁺, 100V): m/z=720.4 [M+NH4]+.

### Example 8: Bexagliflozin Hexyl Carbonate Ester Derivative

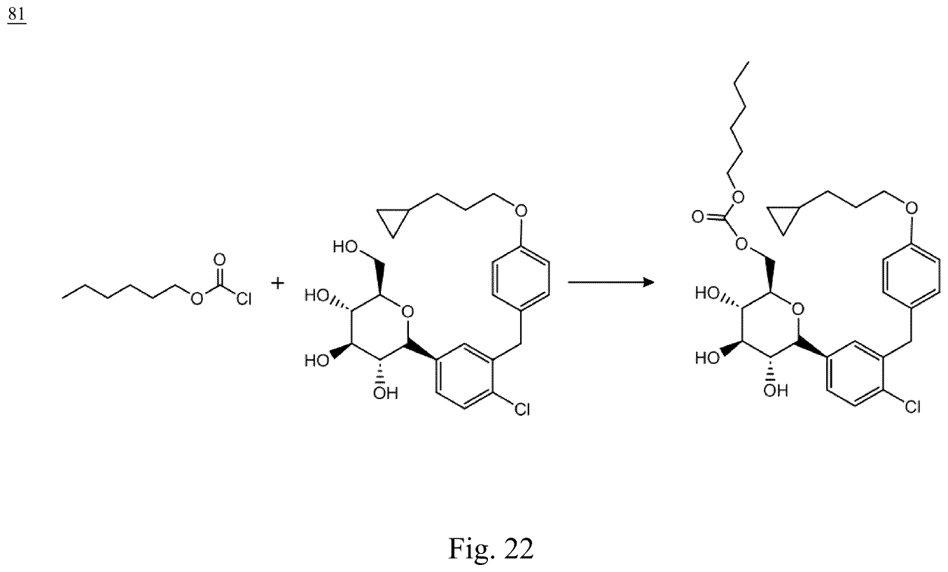

As shown in FIG. 22 (synthesis diagram 81), the synthesis method of bexagliflozin hexyl carbonate ester derivative was as follows.

210 g bexagliflozin was dissolved in 178.63 mL pyridine, 2100 ml tetrahydrofuran and 2100 mL dichloromethane under 10° C. conditions. After complete dissolution, 89.23 g hexyl chloroformate was added and stirred at 10° C. and 1200 rpm for 18-20 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum. The yield of the obtained sample was 31% and the appearance was a white solid.

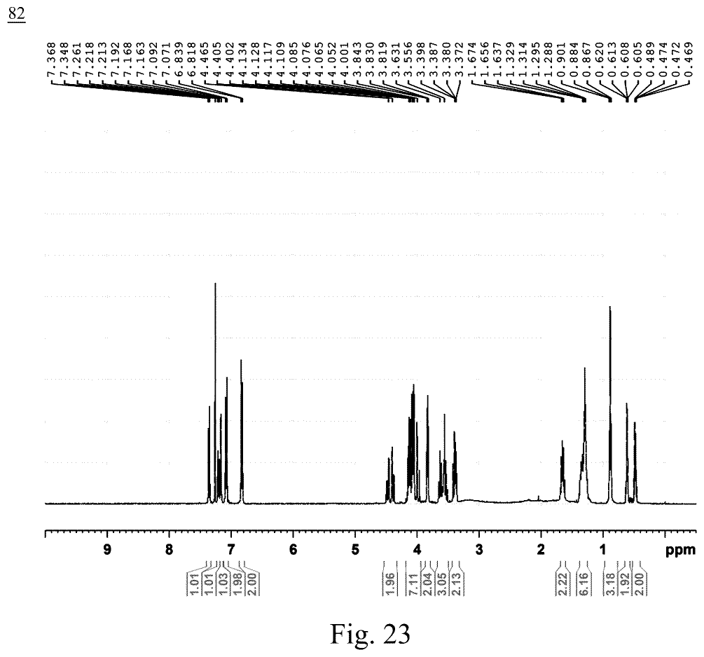

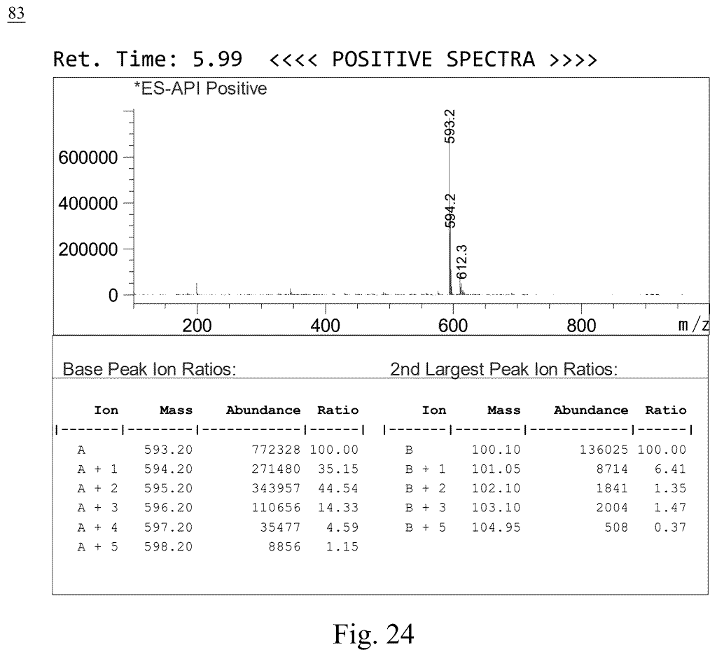

¹H-NMR spectrum and LC-MS spectrum of the obtained sample were represented in FIG. 23 (NMR spectrum 82) and FIG. 24 (LC-MS spectrum 83), respectively, in which ¹H-NMR spectrum represented ¹H-NMR (CDCl₃, 400 MHz, 25° C., zg30 pulse seq.) δ ppm: 0.46-0.49; 0.60-0.62 (4H, CH₂, cyclopropane), 0.86-0.90 (triplet, 3H, CH₃, methyl), 1.19-1.41 (6H, —C₃H₆—, three methylene), 1.55-1.70 (2H, CH₂, methylene), 6.75-6.89 (doublet, 2H, CH, benzyl), 7.01-7.13 (doublet, 2H, CH, benzyl), 7.15-7.19 (doublet, 1H, CH, benzyl), 7.25-7.32 (doublet, 1H, CH, benzyl) and 7.33-7.41 (doublet, 1H, CH, benzyl). Hydrophobic constant was predicted using X Log P3 model methods and X Log P3 was 5.16, which was 2.76 higher than the unmodified Bexagliflozin whose X Log P3 was 2.40. The pKa was 18.82 (ChemDraw). LC-MS was analyzed by the condition of (ESI⁺, 100V): m/z=593.2 [M+H]+.

### Example 9: Bexagliflozin Aromatic Ester Derivative

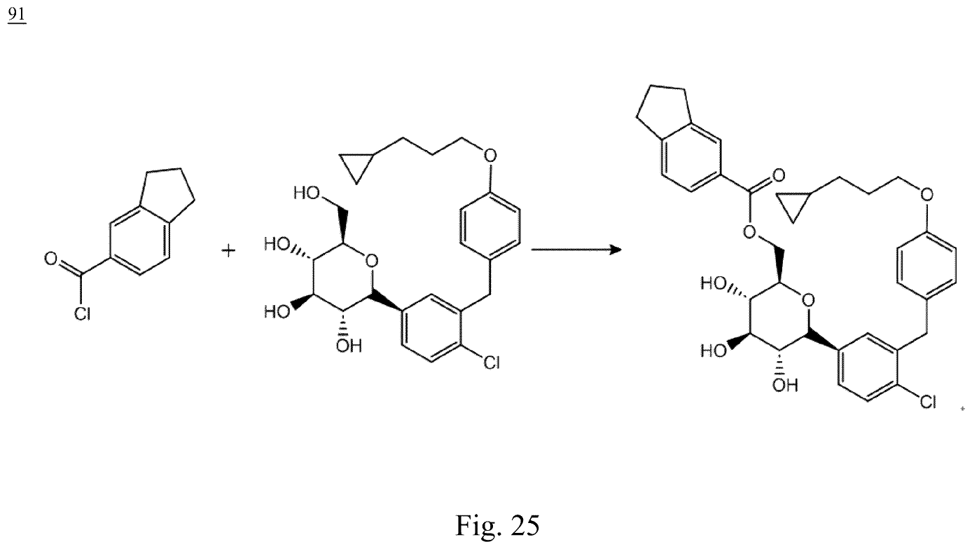

As shown in FIG. 25 (synthesis diagram 91), the synthesis method of bexagliflozin aromatic ester derivative was as follows.

105 g Bexagliflozin and 178 g pyridine was dissolved in 2100 ml tetrahydrofuran and 2100 ml dichloromethane under 10° C. conditions. After complete dissolution, 41 g 2,3-dihydro-1H-indene-5-carbonyl chloride was added and stirred at 0° C.-10° C. and 1200 rpm for 2-6 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum. The yield of the obtained sample was 70% and the appearance was a yellow solid.

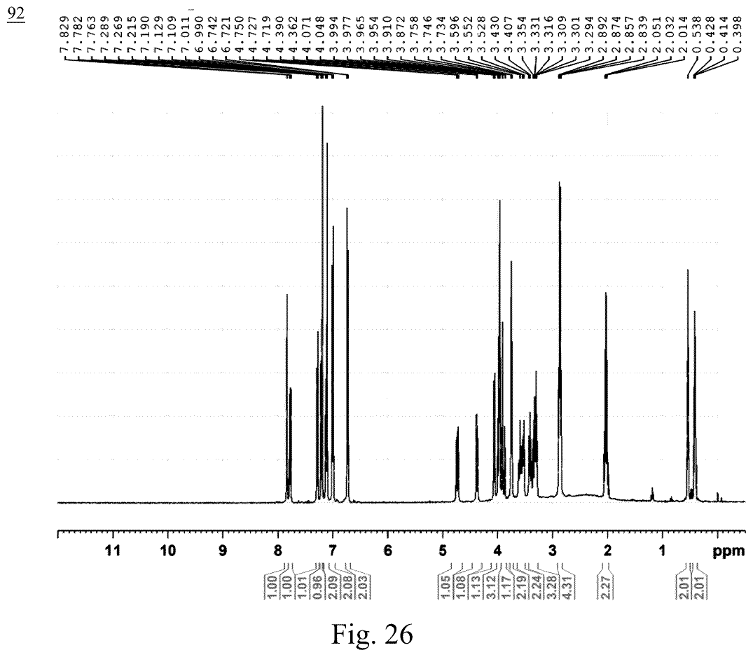

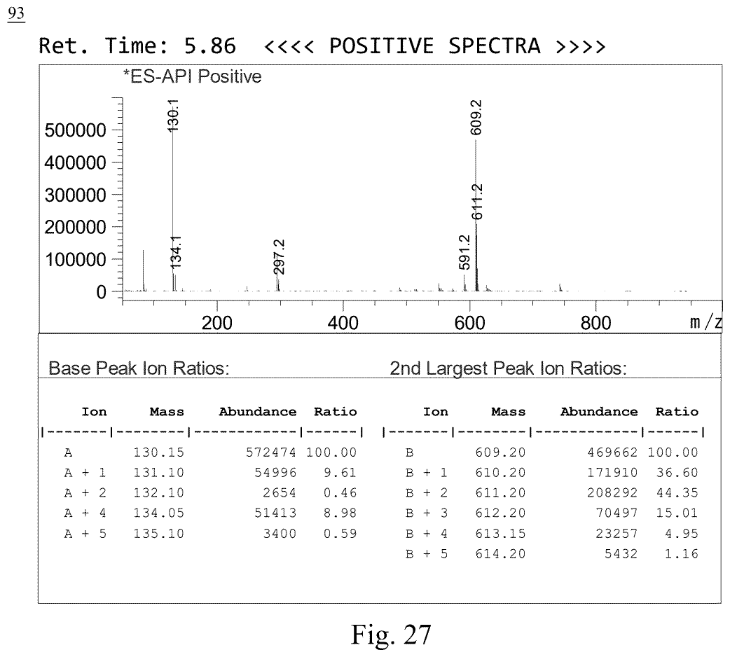

¹H-NMR spectrum and LC-MS spectrum of the obtained sample were represented in FIG. 26 (NMR spectrum 92) and FIG. 27 (LC-MS spectrum 93), respectively, in which ¹H-NMR spectrum represented ¹H-NMR (CDCl₃, 400 MHz, 25° C., zg30 pulse seq.) δ ppm: 0.36-0.42; 0.50-0.56 (4H, CH₂, cyclopropane), 1.98-2.13 (quartet, 2H, CH₂, tetrahydropyran; quintet, 2H, CH₂, indan). 2.80-2.92 (triplet, 4H, two CH₂, indan), 4.65-4.85 (1H, CH, tetrahydropyran), 6.67-6.85 (doublet, 2H, two CH, benzyl), 6.95-7.05 (doublet, 2H, two CH, benzyl), 7.07-7.14 (doublet, 2H, two CH, benzyl), 7.17-7.25 (triplet, 1H, CH, indan), 7.25-7.31 (doublet, 1H, CH, benzyl) and 7.74-7.88 (2H, two CH, indan). Hydrophobic constant was predicted using X Log P3 model methods and X Log P3 was 4.92, which was 2.52 higher than the unmodified Bexagliflozin whose X Log P3 was 2.40. The pKa was 18.86 (Chem Draw). LC-MS was analyzed by the condition of (ESI⁺, 100V): m/z=609.2 [M+H]+.

### Example 10: Other Fatty Acid Ester Derivatives of Dapagliflozin

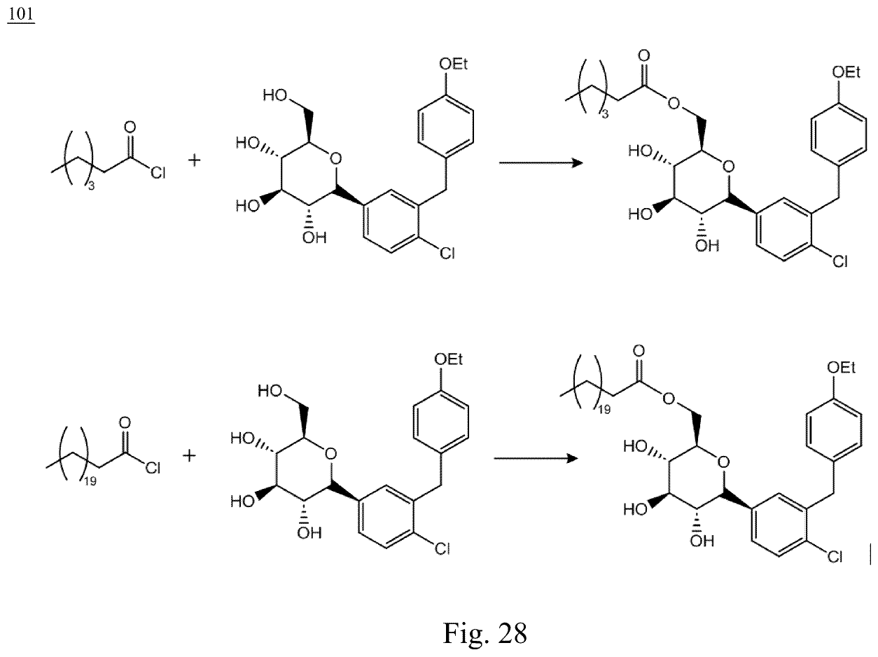

The synthesis method conditions were similar to example 1 and illustrated in FIG. 28 (synthesis diagram 101), in which dapagliflozin was first dissolved in methylene chloride at 10° C. conditions, and then triethylamine was added as catalyst. After that, fatty acid chloride at a molar ratio of 1.3 times that of dapagliflozin was added to the above solution and stirred at 10° C.-20° C. and 1200 rpm for 1-3 hours, the reaction was terminated with half the volume of the organic phase of 10% sodium chloride solution to quench residual acyl chloride. The organic solvent (dichloromethane) was removed under reduced pressure. The product was purified by column chromatography and dried in vacuum and obtained dapagliflozin fatty acid ester derivative products as follows.

TABLE 2
Product Information and XLogP3 Prediction Result
of Dapagliflozin Fatty Acid Ester derivative

| Dapagliflozin | | | Hydrophobic | |
|---|---|---|---|---|
| Fatty Acid Ester | | | Constant | pKa |
| Derivatives | Yield | Appearance | (XLogP3) | (ChemDraw) |
| Hexanoic Acid | 43% | Grayish-white | 4.28 | 16.95 |
| derivative | | solid | | |
| [referred to as | | | | |
| Example 10-1] | | | | |
| Docosanoic Acid | 57% | Grayish-white | 12.95 | 16.95 |
| Derivative | | solid | | |
| [referred to as | | | | |
| Example 10-2] | | | | |

¹H-NMR spectrums and LC-MS spectrums of the obtained samples (Example 10-1 and Example 10-2) were represented below, respectively.

Spectrums and X Log P3 of Example 10-1: Dapagliflozin Hexyl Ester Derivative (Dapagliflozin Hexanoate Derivative)

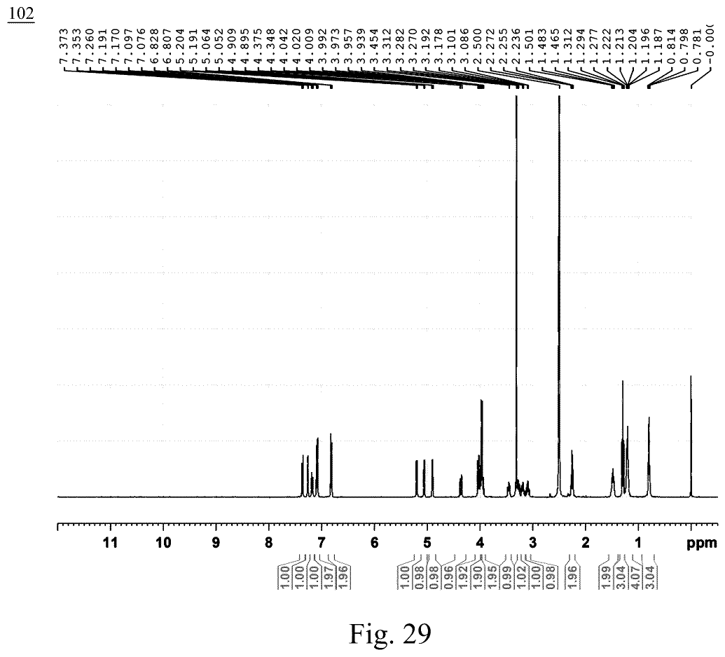

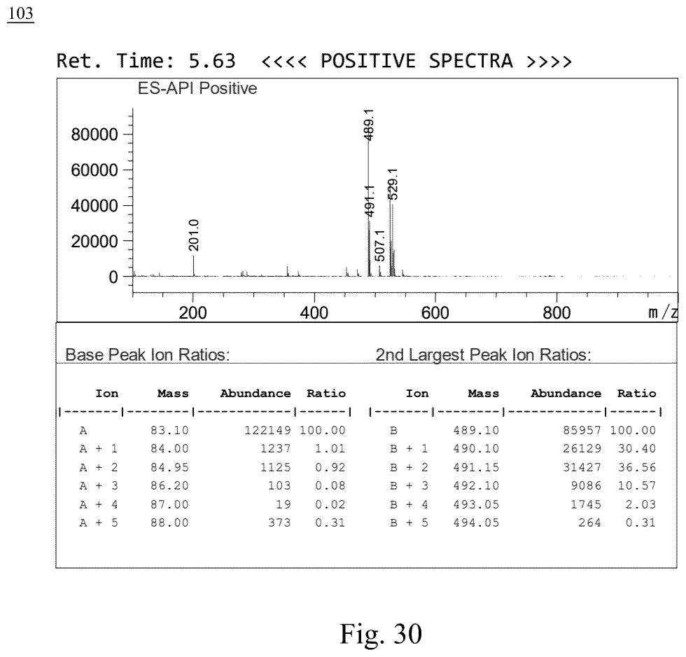

¹H-NMR spectrum and LC-MS spectrum of Example 10-1 were represented in FIG. 29 (NMR spectrum 102) and FIG. 30 (LC-MS spectrum 103), in which ¹H-NMR spectrum represented ¹H-NMR (DMSO-d6, 400 MHz, 25° C., zg30 pulse seq.) δ ppm: 0.71-0.89 (triplet, 3H, CH₃, methyl), 1.10-1.25 (4H, —C₂H₄—, two methylene), 1.26-1.35 (triplet, 3H, CH₃, methyl), 1.40-1.58 (quintet, 2H, CH₂, methylene), 2.19-2.30 (triplet, 2H, CH₂, methylene), 6.75-6.86 (doublet, 2H, CH, benzyl), 7.02-7.15 (doublet, 2H, CH, benzyl), 7.20 (doublet, 1H, CH, benzyl), 7.25 (doublet, 1H, CH, benzyl) and 7.35 (doublet, 1H, CH, benzyl). Hydrophobic constant was predicted using X Log P3 model methods and X Log P3 was 4.28, which was 1.93 higher than the unmodified dapagliflozin whose X Log P3 was 2.35. The pKa was 16.95 (ChemDraw). LC-MS was analyzed by the condition of (ESI⁺, 100V): m/z=529.1 [M+Na]+.

Spectrums and X Log P3 of Example 10-2: Dapagliflozin Docosyl Ester Derivative (Dapagliflozin Docosanoate)

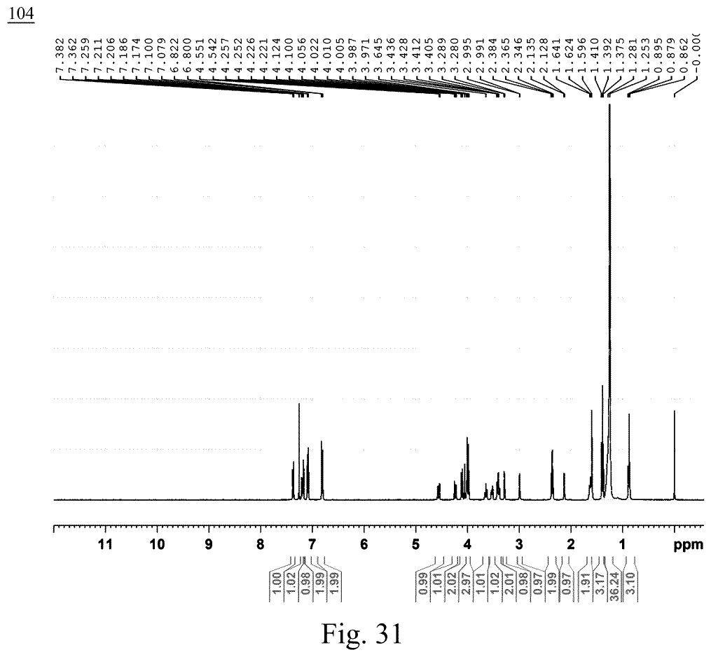

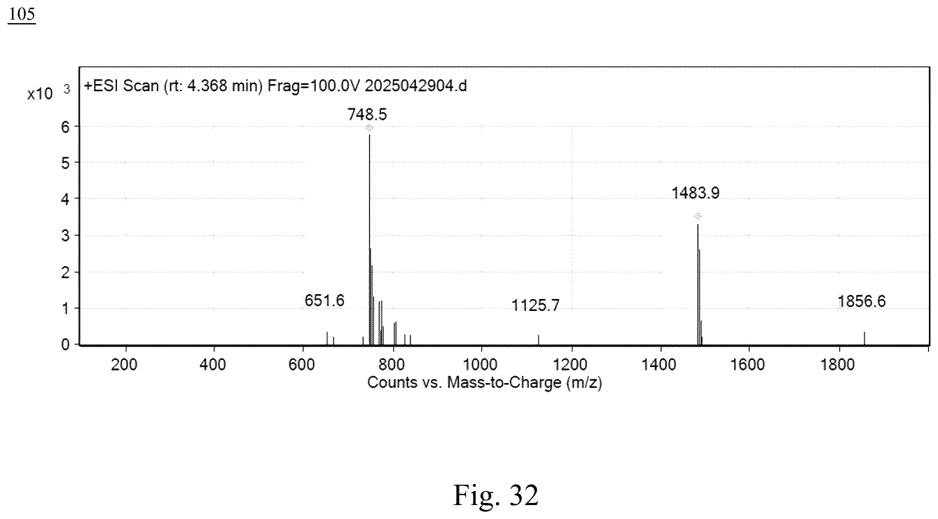

¹H-NMR spectrum and LC-MS spectrum of Example 10-2 were represented in FIG. 31 (NMR spectrum 104) and FIG. 32 (LC-MS spectrum 105), in which ¹H-NMR spectrum represented ¹H-NMR (CDCl₃, 400 MHz, 25° C., zg30 pulse seq.) δ ppm: 0.75-0.95 (triplet, 3H, CH₃, methyl), 1.17-1.35 (36H, —C₁₈H₃₆—, eighteen methylene), 1.36-1.45 (triplet, 3H, CH₃, methyl), 1.55-1.70 (quintet, 2H, CH₂, methylene), 2.29-2.42 (triplet, 2H, CH₂, methylene), 6.75-6.85 (doublet, 2H, CH, benzyl), 7.02-7.15 (doublet, 2H, CH, benzyl), 7.16 (doublet, 1H, CH, benzyl), 7.21 (doublet, 1H, CH, benzyl) and 7.35 (doublet, 1H, CH, benzyl). Hydrophobic constant was predicted using X Log P3 model methods and X Log P3 was 12.95, which was 10.6 higher than the unmodified dapagliflozin whose X Log P3 was 2.35. The pKa was 16.95 (ChemDraw). LC-MS was analyzed by the condition of (ESI⁺, 100V): m/z=748.5 [M+NH4]+.

### 3. Physical Properties Testing of Examples

3.1. Distribution Coefficient Test of N-octanol-water System

To elucidate distribution coefficient of the examples, n-octanol-water system was used as experimental system, and the two-phase system solutions, (1) buffer solution saturated with n-octanol and (2) n-octanol saturated with buffer solution, were required for the n-octanol-water system. First, pH 7.4 buffer solution was prepared, in which 2.40 g of acetic acid, 2.51 g of boric acid and 3.90 g of phosphoric acid were dissolved in water and diluted to 500 mL as a buffer solution. 10 mL buffer solution was taken and adjusted to neutral pH in demand using 0.2M NaOH or 0.2M HCl. After that, the neutral buffer solution was mixed with n-octanol and stirred at 25° C. and 1200 rpm for 1 day to obtain a mixture. Furthermore, the mixture was centrifuged at 10000 rpm for 3 minutes to separate and obtain the buffer solution saturated with n-octanol and the n-octanol saturated with buffer solution.

Then, Log P was measured by using a shake flask method. In detail, n-octanol prepared solution including 0.5 mM analyte was prepared, referring to as “prepared solution” below. Furthermore, 40 μL of the prepared solution was added into 1.96 mL n-octanol saturated with buffer solution (oil phase) and stirred for 30 seconds. Furthermore, 2 mL buffer solution saturated with n-octanol (water phase) was added and stirred at 25° C. and 150 rpm for 24 hours to obtain an analytic mixture. Then, the analytic mixture was centrifuged for 3 minutes (10000 rpm) to separate and obtain a water phase and an oil phase. Then, analyte concentrations in two phases were measured with high-performance liquid chromatography (HPLC), respectively. Distribution coefficient (Log P) was the logarithm of the concentration of the analyte in the oil phase divided by the concentration of the analyte in the water phase.

It should be noted that during the experiment, most measurements were limited by the instrument's limits of quantification (LOQ), so the hydrophobicity could only be inferred through simulated calculations. In detail, except for Examples 2 and 3, where a concentration of the analyte in the water phase was measurable and the actual Log P value could be calculated, the analyte concentrations of Examples 1, 4-9, 10-1 and Example 10-2 detected in the water phase were all below their respective LOQs. Accordingly, the LOQ was used as the water phase concentration for calculations of Log P values. That is, the actual Log P values of those examples should be greater than the calculated values.

For the sake of clarity, some representative Log P values of Examples were represented in Table 3 below.

TABLE 3
Hydrophobic Constant of Example Detected
by N-octanol-water System

| | Hydrophobic Constant |
|---|---|
| Test Sample | (LogP) |
| Example 1 | >4.78 |
| (Dapagliflozin Hexadecyl Ester Derivative) | |
| Example 2 | 5.21 |
| (Dapagliflozin Hexyl Carbonate Ester) | |
| Example 3 | 4.99 |
| (Dapagliflozin Aromatic Ester) | |
| Example 10-2 Dapagliflozin Docosyl Ester | >2.96 |

Taking Example 1 as a reference, the concentration of the analyte measured in the oil phase was 6.49 mg/mL. However, the signal measured in the water aqueous phase was below the LOQ concentration (0.11 μg/mL). Therefore, the LOQ concentration was used for Log P calculation. The ratio of the two concentrations [the concentration of the analyte measured in the oil phase and LOQ concentration] was taken as the logarithmic value, resulting in a Log P of 4.78. Since the analyte concentration of the water phase in the calculation was replaced by LOQ in the calculation, the actual Log P value should be greater than the value (4.78) in Table 3. Consequently, the hydrophobic constant value for Example 1 was expressed as >4.78 in Table 3. This trend of the results also demonstrated that the hydrophobic functional group imparted extremely high hydrophobicity to the derivatives in Examples.

Furthermore, it was observed that the hydrophobic constant (Log P value) of Example 2 in Table 3, 5.21, was close to the predicted value of 5.11 in Table 1-2 obtained using the X Log P3 model; similarly, the hydrophobic constant (Log P value) of Example 3 in Table 3, 4.99, was close to the predicted value of 4.87 in Table 1-2 obtained using the X Log P3 model. The slight discrepancies between the experimental (Log P value) and predicted values (X Log P3) may be attributed to the computational model's inability to perfectly capture all complex intra- and intermolecular interactions. The comparison between Table 1-2 and Table 3 demonstrated that the predicted values in Table 1-2 provided a considerable degree of reference reliability.

### 3.2. Solubility Test of Dapagliflozin Derivatives in Buffer Solution

For testing solubility of examples, dapagliflozin derivatives produced in example 1, example 2, example 3, example 10-1 and example 10-2 were added into 2 mL pH2 HCl/KCl buffer, pH 5 acetate buffer, pH 6 phosphate buffer, pH 7 phosphate buffer, pH 8 phosphate buffer, pH 10 and pH 12 borate buffers, respectively, and stirred with a magnet at 1500 rpm and 25° C. for 24 hours. Furthermore, 1 mL solution was centrifuged at 10000 rpm for 1 minute. The supernatant was filtered with a 0.22 μm nylon filter and analyzed by HPLC (Agilent 1260 HPLC) under the conditions that were a 35° C. C8 column, a 0.1% phosphoric acid aqueous solution and acetonitrile gradient mobile phase, in which a DAD (Diode Array Detector) was used to measure the 210 nm UV absorption at a flow rate of 1 mL/min. Moreover, the solubility literature of dapagliflozin propanediol hydrate was used as a comparative example. The experimental results were shown in the following table 4.

TABLE 4
Solubility of Dapagliflozin Derivatives of Examples 1-3, 10-1 and 10-2 and
Comparative Example (Dapagliflozin Propanediol Hydrate) in Buffer Solution

| Test | Buffer solution | | | | | | |
|---|---|---|---|---|---|---|---|
| sample | pH 2 | pH 5 | pH 6 | pH 7 | pH 8 | pH 10 | pH 12 |
| Example | N.D.^{#} | N.D. | N.D. | N.D. | N.D. | N.D. | N.D. |
| 1 | very | very | very | very | very | very | very |
| | slightly | slightly | slightly | slightly | slightly | slightly | slightly |
| | soluble | soluble | soluble | soluble | soluble | soluble | soluble |
| Example | N.D. | N.D. | N.D. | N.D. | N.D. | <LOQ | <LOQ |
| 2 | very | very | very | very | very | very | very |
| | slightly | slightly | slightly | slightly | slightly | slightly | slightly |
| | soluble | soluble | soluble | soluble | soluble | soluble | soluble |
| Example | N.D. | N.D. | 0.21 | <LOQ | <LOQ | <LOQ | <LOQ |
| 3 | very | very | μg/mL | very | very | very | very |
| | slightly | slightly | very | slightly | slightly | slightly | slightly |
| | soluble | soluble | slightly | soluble | soluble | soluble | soluble |
| | | | soluble | | | | |
| Example | <LOQ | 0.22 | 0.2 | 0.36 | 0.39 | 0.9 | <LOQ |
| 10-1 | very | μg/mL | μg/mL | μg/mL | μg/mL | μg/mL | very |
| | slightly | very | very | very | very | very | slightly |
| | soluble | slightly | slightly | slightly | slightly | slightly | soluble |
| | | soluble | soluble | soluble | soluble | soluble | |
| Example | <LOQ | <LOQ | <LOQ | <LOQ | <LOQ | <LOQ | <LOQ |
| 10-2 | very | very | very | very | very | very | very |
| | slightly | slightly | slightly | slightly | slightly | slightly | slightly |
| | soluble | soluble | soluble | soluble | soluble | soluble | soluble |
| Comparative | 350 | 370 | 320 | 320 | 350 | 340 | 240 |
| Example* | μg/mL | μg/mL | μg/mL | μg/mL | μg/mL | μg/mL | μg/mL |
| | slightly | slightly | slightly | slightly | slightly | slightly | slightly |
| | soluble | soluble | soluble | soluble | soluble | soluble | soluble |

Note 1:
*Data came from literature Pharmaceutics 2023, 15, 1246.
Note 2:
N.D. mean that the absorption signal peak of the synthesized derivative was not detected in the HPLC analysis; <LOQ means that the absorption signal peak of the synthesized derivative was detected in the HPLC analysis but was lower than the detection limit (LOQ) of the analytical method. In this experiment, the LOQ of Dapagliflozin palmitate ester prepared in Example 1 was 0.079 μg/mL; the LOQ of Dapagliflozin hexyl
carbonate ester prepared in Example 2 was 0.045 μg/mL; the LOQ of Dapagliflozin aromatic ester prepared in Example 3 was 0.022 μg/mL; the LOQ of Dapagliflozin hexyl ester prepared in Example 10-1 was 0.05 μg/mL; the LOQ of Dapagliflozin docosyl ester prepared in Example 10-2 was 0.04 μg/mL.
Note 3:
Referring to the definition of solubility ranges in the United States Pharmacopeia (USP) and using milligrams per milliliter (mg/mL) as an example (Drug Des. Devel. Ther. 2014, 8, 1563-1575), the solubility range was classified as follows:
Soluble: ≥33 mg/mL and <100 mg/mL
Sparingly soluble: ≥10 mg/mL and <33 mg/mL
Very sparingly soluble: ≥1 mg/mL and <10 mg/mL
Slightly soluble: ≥0.1 mg/mL and <1 mg/mL
Very slightly soluble: <0.1 mg/mL

As shown in Table 4, it was represented that the solubility range of comparative example (dapagliflozin propanediol hydrate) was about from 240 μg/mL to 370 μg/mL. The dapagliflozin derivatives synthesized in the examples were more hydrophobic than dapagliflozin propylene glycol hydrate, and the solubilities of the dapagliflozin derivatives prepared in Example 1, Example 2, Example 3, Example 10-1 and Example 10-2 in aqueous buffer solution were less than that of the comparative example, indicating that the hydrophobicity was improved by adding the hydrophobic functional group.

### 3.3. Solubility Test of SGLT2 Inhibitor Derivatives in Buffer Solution (Including the Empagliflozin Derivatives of Examples 4-6 and the Bexagliflozin Derivatives of Examples 7-9)

Solubility of examples 4-6 and 7-9 was detected according to the same method as mentioned in point 3.2, and the results were shown in the following table 5 and table 6. In addition, empagliflozin and bexagliflozin compounds were also subjected to the solubility test as comparative examples.

TABLE 5
Solubility of Empagliflozin derivatives of Examples 4-6 and
Comparative Example (Empagliflozin) in Buffer Solution

| Test | Buffer solution | | | | | | |
|---|---|---|---|---|---|---|---|
| Sample | pH 2 | pH 5 | pH 6 | pH 7 | pH 8 | pH 10 | pH 12 |
| Example | <LOQ# | <LOQ | <LOQ | <LOQ | <LOQ | <LOQ | 0.3 |
| 4 | very | very | very | very | very | very | μg/mL |
| | slightly | slightly | slightly | slightly | slightly | slightly | very |
| | soluble | soluble | soluble | soluble | soluble | soluble | slightly |
| | | | | | | | soluble |
| Example | <LOQ | <LOQ | <LOQ | <LOQ | <LOQ | <LOQ | <LOQ |
| 5 | very | very | very | very | very | very | very |
| | slightly | slightly | slightly | slightly | slightly | slightly | slightly |
| | soluble | soluble | soluble | soluble | soluble | soluble | soluble |
| Example | <LOQ | <LOQ | <LOQ | <LOQ | <LOQ | <LOQ | <LOQ |
| 6 | very | very | very | very | very | very | very |
| | slightly | slightly | slightly | slightly | slightly | slightly | slightly |
| | soluble | soluble | soluble | soluble | soluble | soluble | soluble |

| | |
|---|---|
| Comparative | Solubility was 0.5 mg/mL at pH 1.2 to pH 6.3; solubility was 0.4 |
| Example | mg/mL at pH 6.8 to pH 7.5, belonging to slightly soluble. ^{$} |
| (Empagliflozin) | |

Note 1:
The meanings of “N.D.”, “<LOQ” and the classification of solubility were the same as Table 4. In this experiment, the LOQ of Empagliflozin palmitate ester prepared in Example 4 was 0.14 μg/mL; the LOQ of Empagliflozin hexyl carbonate ester prepared in Example 5 was 0.20 μg/mL; the LOQ of Empagliflozin aromatic ester prepared in Example 6 was 0.08 μg/mL.
Note 2:
^{$} Data came from Food and Drug Administration (FDA) document (206073Orig1s000 CLINICAL PHARMACOLOGY AND BIOPHARMACEUTICS REVIEW(S)).

TABLE 6
Solubility of Bexagliflozin derivatives of Examples 7-9 and
Comparative Example (Bexagliflozin) in Buffer Solution

| Test | Buffer solution | | | | | | |
|---|---|---|---|---|---|---|---|
| Sample | pH 2 | pH 5 | pH 6 | pH 7 | pH 8 | pH 10 | pH 12 |
| Example | N.D.^{#} | N.D. | N.D. | N.D. | N.D. | N.D. | N.D. |
| 7 | very | very | very | very | very | very | very |
| | slightly | slightly | slightly | slightly | slightly | slightly | slightly |
| | soluble | soluble | soluble | soluble | soluble | soluble | soluble |
| Example | N.D. | N.D. | N.D. | N.D. | N.D. | N.D. | N.D. |
| 8 | very | very | very | very | very | very | very |
| | slightly | slightly | slightly | slightly | slightly | slightly | slightly |
| | soluble | soluble | soluble | soluble | soluble | soluble | soluble |
| Example | N.D. | N.D. | N.D. | N.D. | N.D. | N.D. | N.D. |
| 9 | very | very | very | very | very | very | very |
| | slightly | slightly | slightly | slightly | slightly | slightly | slightly |
| | soluble | soluble | soluble | soluble | soluble | soluble | soluble |

| | |
|---|---|
| Comparative | Solubility was 0.45 mg/mL at pH 1; solubility was 0.51 mg/mL at |
| Example | pH 4.5; solubility was 0.43 mg/mL at pH 7.6, belonging to slightly |
| (Bexagliflozin)* | soluble. ^{$} |

Note 1:
The meanings of “N.D.”, “<LOQ” and the classification of solubility were the same as Table 4. In this experiment, the LOQ of Bexagliflozin palmitate ester prepared in Example 7 was 0.06 μg/mL; the LOQ of Bexagliflozin hexyl carbonate ester prepared in Example 8 was 0.05 μg/mL; the LOQ of Bexagliflozin aromatic ester prepared in Example 9 was 0.03 μg/mL.
Note 2:
^{$} Data came from FDA document (214373Orig1s000 PRODUCT QUALITY REVIEW(S)).

As shown in Table 5 and Table 6, the solubility ranges of comparative examples (empagliflozin and bexagliflozin) belonged to slightly soluble. The empagliflozin derivatives and bexagliflozin derivatives synthesized in the examples were more hydrophobic than their comparative examples, respectively, and the solubilities of the derivatives prepared in Examples 4-9 in aqueous buffer solution were less than that of the comparative examples, falling within the very slightly soluble range, indicating that the hydrophobicity was improved by adding the hydrophobic functional group.

### 3.4. Melting Point Test of SGLT2 Inhibitor Derivatives

The melting points of the SGLT2 inhibitor derivatives obtained in the examples (“samples” in the following) were measured by a thermogravimetric/thermal differential scanning analyzer (Name: Thermal Analysis System TGA/DSC 3+; Brand: Mettler Toledo). 5-15 mg of the SGLT2 inhibitor derivatives were accurately weighed, added to an aluminum crucible and placed into a heating furnace. Furthermore, the sample heating rate was set to 5° C./min, and the experimental temperature range was set to 25° C.-500° C. A suitable inert gas such as nitrogen was used, and the gas purge rate for both the sample and the balance was set to 40 mL/min. The DSC (Differential Scanning calorimetry) thermogram was obtained through measurement, and the onset temperature of the endothermic energy change peak in the thermogram was used to determine the melting point. The results were shown in Table 7.

TABLE 7
Melting Points of SGLT2 Inhibitor Derivatives

| Test Sample | Melting Point (° C.) |
|---|---|
| Example 1 Dapagliflozin Hexyl Ester | 89.24 |
| Example 2 Dapagliflozin Hexyl Carbonate Ester | 83.93 |
| Example 3 Dapagliflozin Aromatic Ester | N/A* |
| Example 4 Empagliflozin Hexyl Ester | 92.04 |
| Example 5 Empagliflozin Hexyl Carbonate Ester | 73.81 |
| Example 6 Empagliflozin Aromatic Ester | 52.51 |
| Example 7 Bexagliflozin Hexyl Ester | 91.31 |
| Example 8 Bexagliflozin Hexyl Carbonate Ester | 59.86 |
| Example 9 Bexagliflozin Aromatic Ester | N/A* |
| Example 10-1 Dapagliflozin Hexyl Ester | 91.04 |
| Example 10-2 Dapagliflozin Docosyl Ester | 114.36 |
| Comparative Example | |
| Dapagliflozin propanediol monohydrate | 73.58 |
| Empagliflozin | 151.10 |
| Bexagliflozin | 129.08 |

Note 1:
*“N/A” mean no melting point was detected, indicating that no endothermic peak was observed as the temperature gradually increased until decomposition.

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A sodium-glucose cotransporter 2 (SGLT2) inhibitor derivative, comprising a backbone structure and a hydrophobic functional group, wherein the backbone structure comprises a glucoside or a glucoside analog, wherein the glucoside or the glucoside analog is bonded to the hydrophobic functional group via a covalent bond.

2. The SGLT2 inhibitor derivative of claim 1, wherein the backbone structure comprises a structure as below: 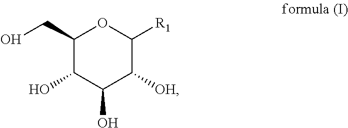
wherein R₁ represents an unsubstituted aryl group, a substituted aryl group, an arylalkyl group, an arylalkylenearyl group, or an heteroaryl group, wherein a substituent of the substituted aryl group comprises C₁-C₆ alkyl, halo, alkoxy, alkenyl or heteroaryl.

3. The SGLT2 inhibitor derivative of claim 2, wherein the glucoside comprises Velagliflozin, Dapagliflozin, Canagliflozin, Empagliflozin, Tofogliflozin, Ipragliflozin, Atigliflozin, Bexagliflozin or Janagliflozin.

4. The SGLT2 inhibitor derivative of claim 1, wherein the glucoside analog comprises a first glucoside analog, a second glucoside analog or a third glucoside analog, wherein the first glucoside analog represents oxygen-containing bridge skeleton formed in glucose six-membered ring of the glucoside, the second glucoside analog represents that oxygen atom in the glucose six-membered ring of the glucoside is replaced by sulfur, and the third glucoside analog represents carbon 6 in the glucoside is replaced by a methylthio group.

5. The SGLT2 inhibitor derivative of claim 4, wherein the first glucoside analog comprises a structure as below: 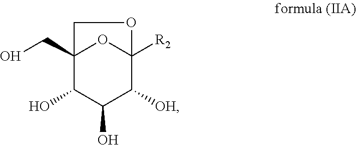
wherein R₂ represents an unsubstituted aryl group, a substituted aryl group, an arylalkyl group, an arylalkylenearyl group, or a heteroaryl group, wherein a substituent of the substituted aryl group comprises C₁-C₆ alkyl, halo, alkoxy, alkenyl or heteroaryl.

6. The SGLT2 inhibitor derivative of claim 5, wherein the first glucoside analog comprises Ertugliflozin or Henagliflozin.

7. The SGLT2 inhibitor derivative of claim 4, wherein the second glucoside analog comprises a structure as below: 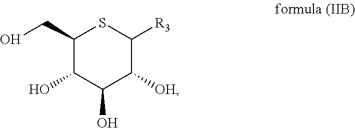
wherein R₃ represents an unsubstituted aryl group, a substituted aryl group, an arylalkyl group, an arylalkylenearyl group, or an heteroaryl group, wherein a substituent of the substituted aryl group comprises C₁-C₆ alkyl, halo, alkoxy, alkenyl or heteroaryl.

8. The SGLT2 inhibitor derivative of claim 7, wherein the second glucoside analog comprises Luseogliflozin.

9. The SGLT2 inhibitor derivative of claim 4, wherein the third glucoside analog comprises a structure as below: 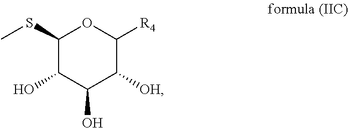
wherein R₄ represents an unsubstituted aryl group, a substituted aryl group, an arylalkyl group, an arylalkylenearyl group, or an heteroaryl group, wherein a substituent of the substituted aryl group comprises C₁-C₆ alkyl, halo, alkoxy, alkenyl or heteroaryl.

10. The SGLT2 inhibitor derivative of claim 9, wherein the third glucoside analog comprises Sotagliflozin.

11. The SGLT2 inhibitor derivative of claim 1, wherein the hydrophobic functional group comprises an aliphatic group, an aromatic ring group or a combination thereof, wherein the aliphatic group has at least 6 carbon atoms.

12. The SGLT2 inhibitor derivative of claim 11, wherein the hydrophobic functional group is bonded at oxygen atom of C2, C3, C4, or C6 of the glucoside or the glucoside analog.

13. The SGLT2 inhibitor derivative of claim 12, wherein when the backbone structure is the glucoside, the SGLT2 inhibitor derivative has a structure of formula (III): 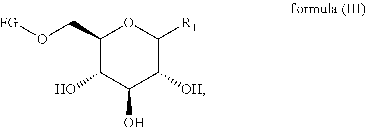
wherein when the backbone structure is the glucoside analog, the SGLT2 inhibitor derivative has a structure of formula (IVA), formula (IVB) or formula (IVC): 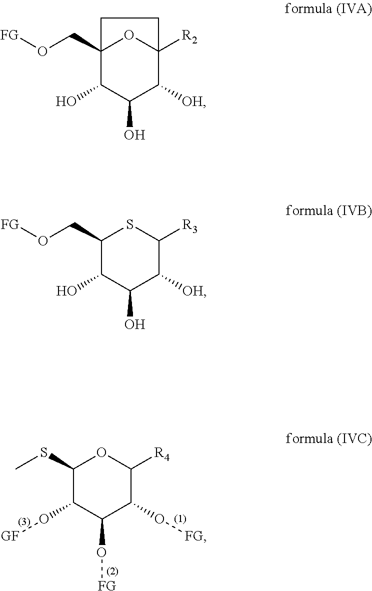
wherein R₁, R₂, R₃ and R₄ represent an unsubstituted aryl group, a substituted aryl group, an arylalkyl group, an arylalkylenearyl group, or an heteroaryl group, respectively, a substituent of the substituted aryl group comprises C₁-C₆ alkyl, halo, alkoxy, alkenyl or heteroaryl, and FG represents the hydrophobic functional group and is bonded to O at position (1), position (2), position (3) or a combination thereof.

14. The SGLT2 inhibitor derivative of claim 11, wherein the aliphatic group comprises hexyl, octyl, decyl, dodecyl, tetradecyl, hexadecyl, octadecyl, icosyl, docosyl, tetracosyl, caproyl, capryloyl, decanoyl, dodecanoyl, tetradecanoyl, hexadecanoyl, octadecanoyl, icosanoyl, docosanoyl, tetracosanoyl or a combination thereof.

15. The SGLT2 inhibitor derivative of claim 11, wherein the aromatic ring group comprises cinnamoyl, phenylacetyl, indanyl, salicyl, anthranilyl, benzoyl or a combination thereof.

16. The SGLT2 inhibitor derivative of claim 1, wherein the covalent bond comprises an ester bond, an acetal bond or a ketal bond.

17. The SGLT2 inhibitor derivative of claim 16, wherein the ester bond is a carbonate ester bond, a fatty acid ester bond, a phosphodiester bond, an aromatic ester bond or a carbamate bond.

18. The SGLT2 inhibitor derivative of claim 1, wherein the SGLT2 inhibitor derivative has a hydrophobic constant of more than 3.

19. The SGLT2 inhibitor derivative of claim 18, wherein the hydrophobic constant is calculated by X Log P3 or measured by a distribution coefficient test of n-octanol-water system.

20. The SGLT2 inhibitor derivative of claim 1, wherein a solubility of the SGLT2 inhibitor derivative is more than 0 and less than 0.1 mg/mL in pH 6 phosphate buffer.
